(19) United States
(12) Patent Application Publication
(10) Pub. No.: US 2007/0020618 A1
(43) Pub. Date: Jan. 25, 2007

(54) Process to study changes in gene expression in T lymphocytes

(75) Inventors: Yatindra Prashar, Monmouth Junction, NJ (US); Sherman M. Weissman, New Haven, CT (US)

Correspondence Address: MORGAN LEWIS & BOCKIUS LLP, 1111 PENNSYLVANIA AVENUE NW, WASHINGTON, DC, 20004, US

(73) Assignee: Gene Logic Inc.

(21) Appl. No.: 11/408,059

(22) Filed: Apr. 21, 2006

Related U.S. Application Data
(63) Continuation of application No. 09/674,607, filed on Nov. 14, 2001, now abandoned.
(60) Provisional application No. 60/084,329, filed on May 5, 1998.

(51) Int. Cl. C12Q 1/70; C12Q 1/68

(52) U.S. Cl. 435/5; 435/6

(57) ABSTRACT

Methods are disclosed to identify T lymphocyte genes that are differentially expressed upon exposure to a pathogen (viral or bacterial), immunogen, antigen, or in a sterile inflammatory disease, autoimmune disease, immunodeficiency disease, lymphocytic cancers, or graft versus host rejection. The method involves the preparation of a gene expression profile of a T lymphocyte population exposed to a pathogen or isolated from a subject having one of the aforementioned pathologies and comparing that profile to a profile prepared from quiescent T lymphocytes. The present invention is useful for identifying cytokine genes, genes encoding cell surface receptors and genes encoding intermediary signalling molecules. Related methods for identifying therapeutic or prophylatic immunomodulatory agents are present. Articles of manufacture are disclosed that comprise selected grouping of nucleic acids, affixed to a solid support, that correspond to genes that are differentially expressed in various populations or subpopulations of T lymphocytes at variations stages of T cell differentiation, in quiescent versus activated T lymphocytes or normal versus diseased T lymphocytes.

This application is based on provisional application 60/084,329, filed May 5, 1998, which is incorporated herein by reference in its entirety. This application is related to application Ser. No. 08/510,032 and Ser. No. 08/688,514, both of which are herein incorporated by reference in their entirety. This application is also related to provisional applications Ser. No. 60/056,844 (Atty. Dkt. No. 044574-5003) and Ser. No. 60/056,861 (Atty. Dkt. No. 044574-5014) which are herein incorporated by reference in their entirety.

### TECHNICAL FIELD

This invention relates to compositions and methods that are useful to identify agents that modulate the response of T lymphocytes (T cells) to a variety of foreign antigens and superantigens, and to modulate the role of T lymphocytes in immune deficiency diseases, cancer, tissue transplantation and immune disorders. The invention also relates to compositions and methods of identifying agents that modulate the differentiation of prothymocytes into specific T lymphocyte subpopulations.

### BACKGROUND OF THE INVENTION

The immune system is organized to solve the problems of rapid and specific recognition of an enormous number of potential antigens by cell-to-cell collaboration and by clonal expansion of cells specific for a given antigen. The immune response depends on T lymphocytes acting at various steps. One significant cell-to-cell collaboration in which T lymphocytes take part is the interaction between antigen presenting cells such as macrophages. Another significant cell-to-cell collaboration is between T lymphocytes (helper T lymphocytes) and antibody producing B cells. In both of these collaborations, the ability of T lymphocytes to respond in an antigen-specific manner is central to the strategy of the immune system.

Besides recognizing foreign protein antigens, T lymphocytes also retain a functional memory of virtually all self-proteins which enables then to distinguish between self and nonself antigens. T lymphocytes are born in the bone marrow and mature in the thymus where they learn tolerance to self antigens. It is this ability to recognize self from nonself antigens that allow T lymphocytes to recognize and respond to potential invaders.

T lymphocytes are divided into subpopulations on the basis of function and phenotypic markers. Different T lymphocyte subpopulations function to help in antibody formation (T helper cells, T_{H}), to kill target cells (T-cytotoxic cells, T_{CTL}), to induce inflammation (T-delayed hypersensitivity cells, T_{DTH}), and to inhibit immune responses (T-suppressor cells, T_{S}). T helper cells are further divided into two major subpopulations: T_{H1} and T_{H2}. T_{H1} cells function primarily as helper cells for induction of B-cell proliferation and differentiation to IgG-producing plasma cells; whereas T_{H2} cells produce factors (e.g., interleukins) that induce B cells to differentiate and produce IgE and IgA. T_{H1} cells may further differentiate into T_{DTH} cells that are responsible for the inflammatory effects of T lymphocytes (e.g., delayed hypersensitivity, cytotoxicity) and secrete inflammatory mediators, such as lymphotoxin and interferon-γ (IFN-γ). T_{H2} cell products may also stimulate differentiation of other white blood cells such as eosinophils and basophils.

T lymphocytes respond to antigen by cell activation and proliferation. This activation and proliferation coincides with a release of many effector molecules (e.g., interleukins) that activate or deactivate other lymphocytes, contribute to immune-mediated inflammation (lymphokines), or interact with other cell types. For instance, interleukins produced by T_{H} cells are required to induce activation and differentiation of B cells. At least 20 different interleukins have now been identified and are numbered from interleukin-1 (IL-1) to interleukin-20 (IL-20). T_{H1} cells produce IL-2, IFN-γ and tumor necrosis factor in response to antigen stimulation, whereas T_{H2} cells synthesize IL-4, IL-5 and IL-6. Activation and differentiation of T_{CTL} cells result in the appearance of cytoplasmic granules (secretory lysosomes). The T_{CTL} granules contain perforin, granzymes, and proteoglycans that act to lyse or kill other cells. Therefore, the T lymphocyte population not only contains a variety of effector cells, but also is the master regulator of the immune system; the T lymphocyte is the director of the immunological orchestra of cells and proteins. T lymphocytes function to turn off or on other cells in the immune system via T suppressor cells, T helper cells and T contrasuppressor cells. STEWART SELL, IMMUNOLOGY, IMMUNOPATHOLOGY & IMMUNITY 30-33 (1996). T lymphocytes regulate (directly or indirectly) virtually all aspects of host immune resistance to infection, which include macrophage activity, antibody synthesis, synthesis of other inflammatory proteins, generation of inflammatory cells in the bone marrow and their recruitment into the circulation, and differentiation of other effector and regulatory lymphocytes.

The release of effector molecules and other changes in T lymphocytes during activation are preceded by changes in the expression levels of many genes. The regulation of expression levels of numerous T lymphocyte genes has been an area of extensive study (see Ullman et al., (1990) Ann. Rev. Immunol., 8: 421-452 and Kelly et al., (1995) Curr. Opin. Immunol. 7: 327-332). The present inventor has utilized the analysis of differential gene expression on a more global level, as opposed to the analysis of the expression levels of individual RNA species, to provide simultaneous, near-quantitative information about the levels of gene expression for the multitude of genes whose expression levels are modulated during T lymphocyte activation.

### SUMMARY OF THE INVENTION

While the role(s) of T lymphocytes and T lymphocyte subpopulations in cancer, infectious disease, and autoimmune and immunodeficiency disorders have been subject of intense study, the techniques of differential gene expression have not been exploited to identify therapeutic or prophylactic agents that modulate the response of T lymphocytes in these various roles. The present invention provides an approach to systematically assess the transcriptional response from lymphocytes activated through contact with a pathogen or from T lymphocytes isolated from a subject with an infectious diseases, immune disorder, GVHD or neoplasm involving T lymphocytes.

One preferred embodiment relates to a method to identify a therapeutic or prophylactic agent that modulates the response of a T lymphocyte population to an antigen. This method comprises the steps of (1) preparing a first gene expression profile of a quiescent T lymphocyte population; (2) preparing a second gene expression profile of a T lymphocyte population exposed to the antigen; (3) treating the exposed T lymphocyte population with the agent; (4) preparing a third gene expression profile of the treated T lymphocyte population; (5) comparing the first, second and third gene expression profiles; and (6) identifying agents that modulate the response of a lymphocyte population to the antigen.

The invention also relates to a method to identify a therapeutic agent that modulates a T lymphocyte population found in a subject having a sterile inflammatory disease, autoimmune disorder, immunodeficiency disease, cancer or graft-versus-host disease (GVHD). This method comprises the steps of: (1) preparing a first gene expression profile of a T lymphocyte population in a subject having the sterile inflammatory disease, autoimmune disorder, immunodeficiency disease, cancer, or GVHD; (2) treating the T lymphocyte population with the agent; (3) preparing a second gene expression profile of the treated T lymphocyte population; (4) comparing the first and second gene expression profiles with a gene expression profile of a normal T lymphocyte population; and (5) identifying an agent that modulates a T lymphocyte population found in a subject having a sterile inflammatory disease, autoimmune disorder, immunodeficiency disease, cancer, or GVHD.

In yet another aspect, the present invention relates to a method of diagnosing a sterile inflammatory disease, autoimmune disorder, immunodeficiency disease, cancer, or GVHD in a subject. The method comprises the steps of: (1) preparing a first gene expression profile of a T lymphocyte population from the subject; (2) comparing the first gene expression profile to at least one second gene expression profile from a T lymphocyte population from a subject having a sterile inflammatory disease, autoimmune disorder, immunodeficiency disease, cancer, or GVHD and to a third gene expression profile of a normal T lymphocyte population; and (3) determining if the subject has a sterile inflammatory disease, autoimmune disorder, immunodeficiency disease, cancer, or GVHD.

Another aspect of the present invention relates to a method of identifying therapeutic or prophylactic compounds that modulate a T lymphocyte population that arises from a genetic defect or mutation. This method comprises the steps of: (1) preparing a first gene expression profile of a T lymphocyte population that arises from the genetic defect; (2) treating the first T lymphocyte population with the agent; (3) preparing a second gene expression profile of the treated T lymphocyte population; (4) comparing the first and second gene expression profiles with a gene expression profile of a normal T lymphocyte population; and (5) identifying agents that modulate a T lymphocyte population that arises from a genetic defect.

In contemplated variations of the foregoing methods, relevant data from, e.g., the first or second (or third) gene expression profile (e.g., as shown by the positions and intensities of bands on a gel or the quantity, intensity and relative elution position from a column) may be compared with corresponding relevant data with data that has been stored in an electronic or computer-readable format. For example, a data base of the gene expression profile of quiescent T cells may be used to compare the gene expression profile of T cells that have been activated by exposure to any particular antigen. Similarly, a gene expression profile of a T cell population exposed to an agent that is being evaluated as a candidate agent to modulates the response of a T lymphocyte population to an antigen can be compared to a data base of the gene expression characteristics of a gene expression profile of a T lymphocyte population exposed to that antigen.

Thus, the present invention also relates to a method to identify a therapeutic or prophylactic agent that modulates the response of a T lymphocyte population to an antigen, comprising the steps of: (1) preparing a first gene expression profile of a T lymphocyte population exposed to the antigen; (2) treating the exposed T lymphocyte population with the agent; (3) preparing a third gene expression profile of the treated T lymphocyte population; (4) comparing the first and second gene expression profiles with a data base containing the corresponding gene expression profile information from a quiescent T lymphocyte population not contacted with the antigen; and (6) identifying agents that modulate the response of a lymphocyte population to the antigen.

The present invention also relates to a method to identify a therapeutic or prophylactic agent that modulates the response of a T lymphocyte population to an antigen, comprising the steps of: (1) preparing a first gene expression profile from a T lymphocyte population that has been exposed to the antigen and with the candidate therapeutic or prophylactic agent; and (2) comparing the first gene expression profile with a data base containing the corresponding gene expression profile information from a quiescent T lymphocyte population not contacted with the antigen and, as appropriate, also comparing the first gene expression profile with the corresponding gene expression profile information from a T lymphocyte population that was contacted with the antigen but not contacted with the candidate therapeutic or prophylactic agent; and (3) identifying agents that modulate the response of a lymphocyte population to the antigen.

In a similar fashion, it is contemplated that the gene expression profile information from the “first” gene expression profiles in the methods summarized above, i.e., from quiescent or from diseased but untreated T lymphocyte populations, can reside in a data base to which the gene expression profile information of the “second” gene expression profile may be compared.

Such data bases also reflect another aspect of the present invention and these data bases may contain data based on one or more separately prepared profiles and further may reflect averaged or normalized or otherwise manipulated information. When comparisons are made using data that reflects the average of separately prepared profiles, an average prepared from two separate profiles in preferred, more preferably from three or four such profiles, and most preferably from five or more such profiles. One skilled in the art will know how to prepare and manipulate the information in such data bases in order to maximize the practical value of the data contained therein.

In an article of manufacture aspect, the present invention relates to a grouping of nucleic acids affixed to a solid support, said nucleic acids preferably represent the genes or fragments of genes (or corresponding cDNAs or RNAs) whose expression levels are modulated in a T lymphocyte population that arises, e.g., from exposure to an antigen; are modulated in a T lymphocyte population found in a subject having a sterile inflammatory disease, autoimmune disorder, immunodeficiency disease, cancer, or GVHD; or are modulated in a T lymphocyte population arising from a genetic defect.

In yet another aspect, the present invention relates to an isolated nucleic acid molecule comprising the structure: R-X-R′, wherein X is the novel DNA sequence (or corresponding RNA sequence) or novel portions thereof as identified in SEQ ID NOS. 16, 22, 24, 25, 31, 33 or 34; and wherein R and R′ are sequences contiguous with X in nucleic acid fragments which specifically hybridize with X.

### BRIEF DESCRIPTION OF THE DRAWINGS

FIG. 1 presents autoradiograms of the expression profiles generated from cDNAs made with RNA isolated from quiescent Jurkat cells treated with either 12-O-tetradecanoylphorbol-13-acetate (TPA) and phytohemagglutinin (PHA) or ionomycin and TPA.

| Primer Set | Lanes | Primer | N1 | N2 |
|---|---|---|---|---|
| 1 | 1, 2 | 8.3 | A | C |
| 2 | 3, 4 | 8.4 | C | T |
| 3 | 5, 6 | 8.5 | G | C |
| 4 | 7, 8 | 8.6 | C | C |
| 5 | 9, 10 | 9.2 | G | A |
| 6 | 11, 12 | 9.3 | A | A |
| 7 | 13, 14 | 9.4 | C | G |
| 8 | 15, 16 | 9.5 | A | T |
| 9 | 17, 18 | 10.2 | A | G |
| 10 | 19, 20 | 10.3 | C | A |
| 11 | 21, 22 | 10.4 | G | G |
| 12 | 23, 24 | 10.5 | G | T |

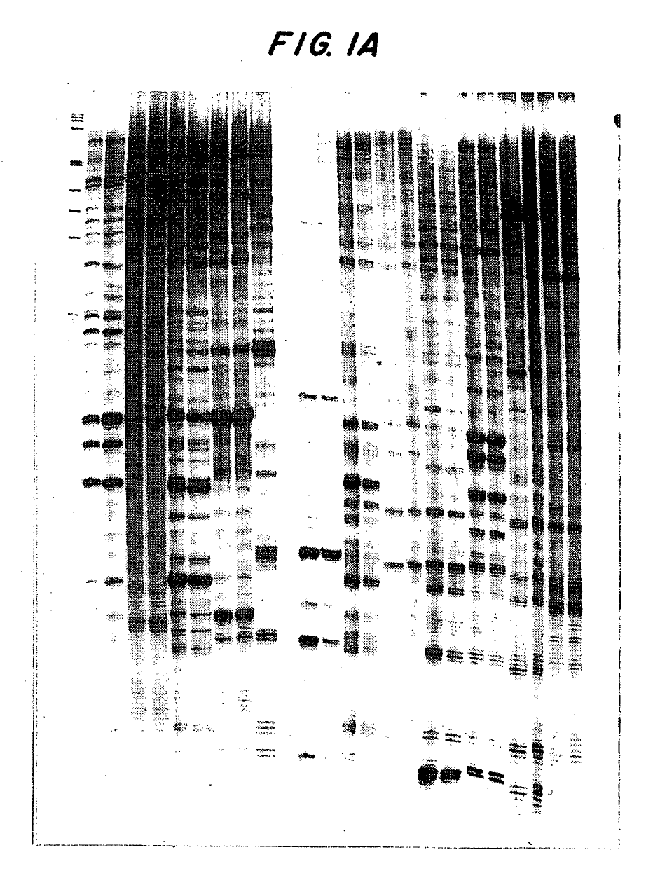

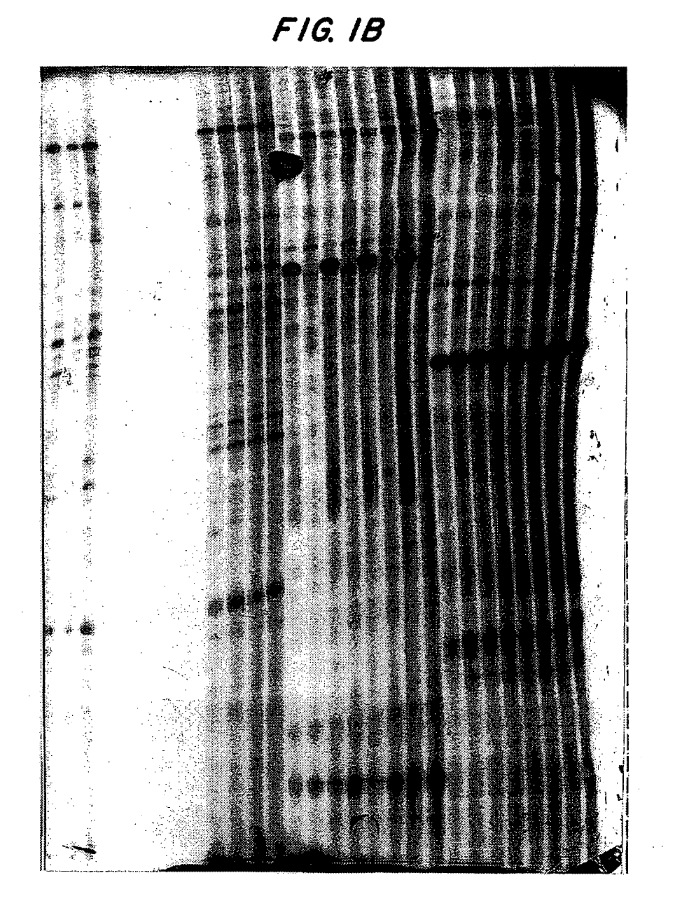

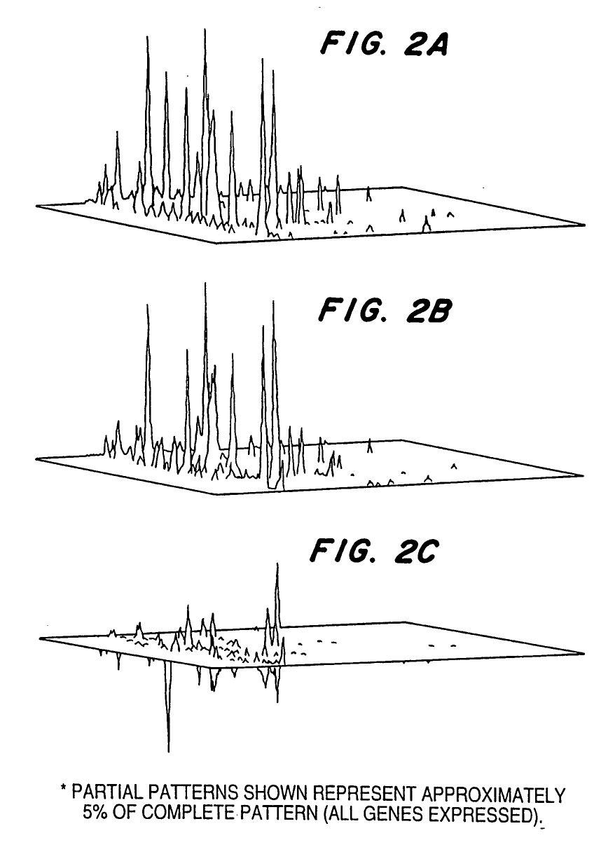

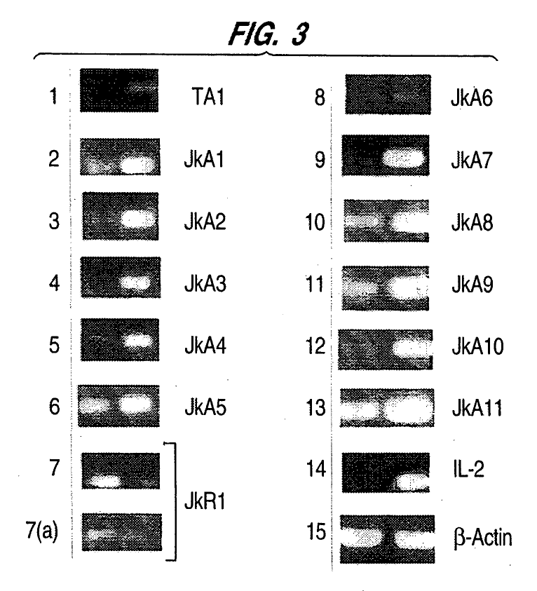

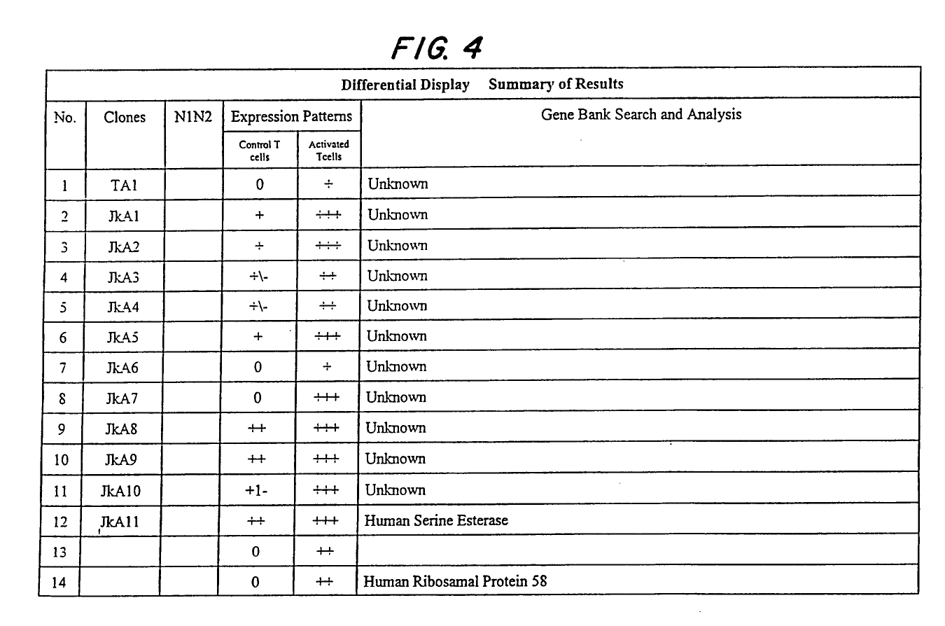

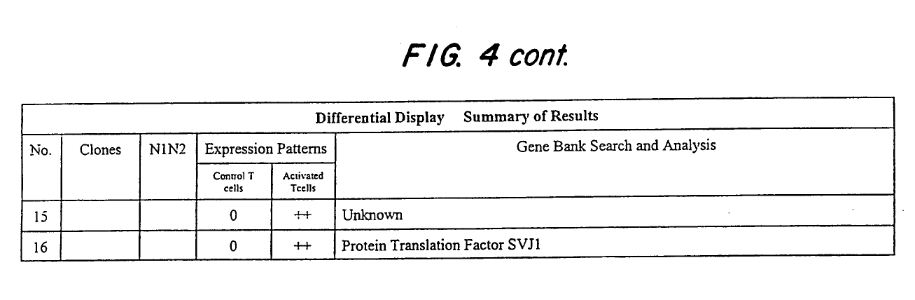

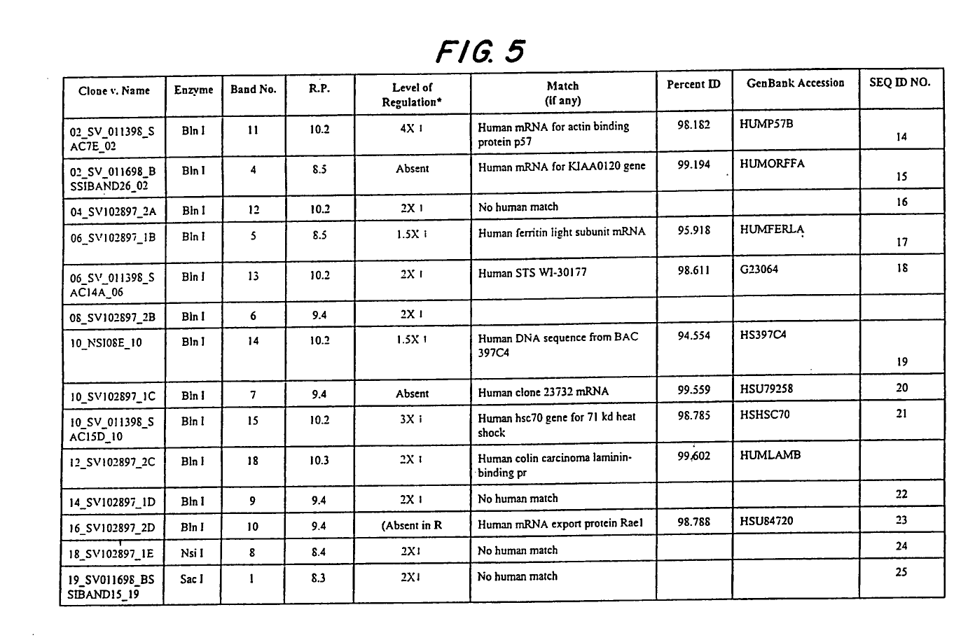

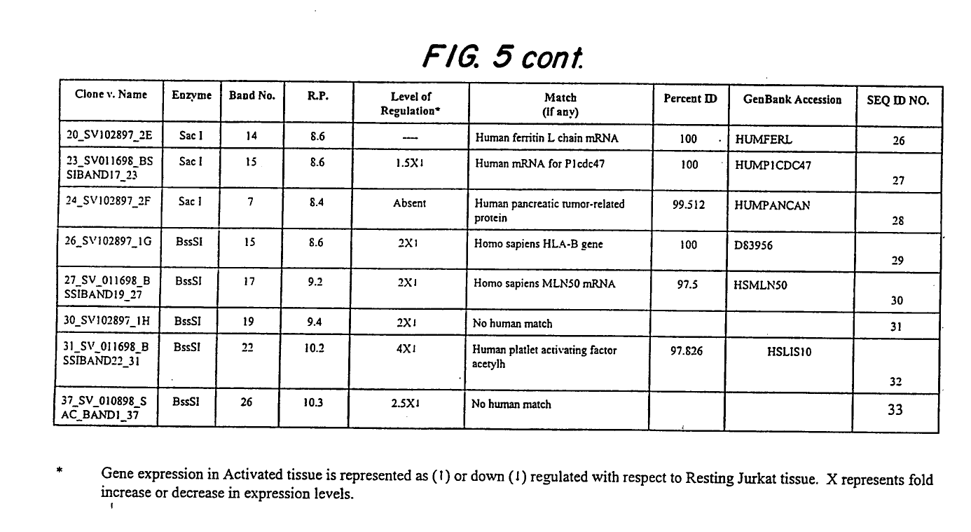

(A) All possible 12 anchoring oligo d(T₁₈)n1, n2 were used to generate a complete expression profile for the restriction enzyme Bgl II. Expression profiles were also generated, and yielded analogous results, using all possible 12 anchoring oligo d(T₁₈)n1, n2 for each of the following restriction enzymes: XbaII, SpeI, NcoI, Hind III, BamHI and XbaI. (B) FIG. 1B is an autoradiogram of dilutions using RP 8.6 wherein the cDNAs have been digested with Hind III.

FIG. 2 is a three dimensional and generalized graphical representation of gene expression in (a) quiescent T lymphocytes; (b) gene expression in activated T lymphocytes; and (c) the differences in gene expression between quiescent and activated T lymphocytes.

FIG. 3 represents quantitative differences of specific cDNA bands from display gels of quiescent versus activated T lymphocytes (Jurkat cells). Clone TA1 (SEQ ID NO.1) JkA1 (SEQ ID NO.2) TA1 (SEQ ID NO.1) JkA2 (SEQ ID NO.3), JkA3 (SEQ ID NO.4) JkA4 (SEQ ID NO.5) JkA5 (SEQ ID NO.6) JkA6 (SEQ ID NO.7) JkA7 (SEQ ID NO.8) JkA8 (SEQ ID NO.9) JkA9 (SEQ ID NO.10) JkA10 (SEQ ID NO.11) JkA11 (SEQ ID NO.12) JKR1 (SEQ ID NO.13)

In each panel, the left lane is a cDNA band from untreated T lymphocytes and the right lane is the corresponding cDNA band from activated T lymphocytes. In panel 7 (not panel 7(a)), peripheral blood T lymphocyte RNA was used for RT-PCR.

FIG. 4 is a table of the cDNA bands of FIG. 3 that correspond to mRNA species that are differentially expressed in the quiescent Jurkat cells and in activated Jurkat cells.

FIG. 5 presents summary data for mRNAs that are differentially expressed in activated versus quiescent T cells and the identity of cDNAs corresponding to said mRNAs. Although not represented in the figure, the inventors have identified yet another sequence (SEQ ID NO.34) that corresponds to a differentially expressed mRNA.

### MODES OF CARRYING OUT THE INVENTION

General Description

The response of T lymphocytes to antigens, superantigens, allografts and xenografts, T lymphocyte function in autoimmune and immunodeficiency disorders, as well as T lymphocyte neoplasms, is a subject of primary importance in view of the need to find ways to modulate the immune response. Similarly, the response of T lymphocytes to agonists (pro-inflammatory molecules), such as IL-2, is a subject of great importance in view of the need to find better methods of controlling inflammation brought about by various disease states.

One means of assessing the response of T lymphocytes to antigens, as well as determining differences between diseased lymphocytes versus normal, is to measure the ability of T lymphocytes to synthesize specific RNA de novo either upon contact with an antigen, allergen or pathogen or the state of RNA message in T lymphocytes found in different T lymphocyte disorders.

The following discussion presents a general description of the invention as well definitions for certain terms used herein.

Definitions

As used herein, “T lymphocyte population”, also referred to as a “T cell population”, refers to any population of T lymphocytes obtained from a variety of sources, such as mammalian (e.g., human) spleen, tonsils and peripheral blood. See Lewis et al., (1988) Proc. Natl. Acad. Sci., 85: 9743-9747. T-cell clones, such as a Jurkat cell line, may also be used. Such T lymphocyte clones are numerous and commonly available (see Research Monographs in Immunology, eds. von Doehmer, H. and Haaf, V.; Section D: “Human T-Cell Clones”, vol. 8, pgs. 243-333; Elsevier Science Publishers, N.Y. [1985]).

As used herein, “quiescent T lymphocytes” refers to resting T lymphocytes that have not been activated by exposure to an activating agent, pathogen, mitogen, immunogen, allergen, antigen or superantigen or any other agent that induces a change in T lymphocyte mRNA expression.

As used herein, “activated T lymphocytes” refers to T lymphocytes which have been exposed to an activating agent, pathogen, mitogen, immunogen, allergen, antigen, superantigen or any other agent that induces a change in T lymphocyte mRNA expression.

As used herein, “T_{H1}” refers to the subpopulation of T helper cells which secrete IL-2 and interferon γ (IFN-γ), but not IL-4. T_{H1} cells also secrete IL-3 and granulocyte-monocyte colony-stimulating factor (GM-CSF).

As used herein, “T_{H2}” refers to the subpopulation of T helper cells which secrete IL-4, IL-5, but not IL-2 or IFN-γ. T_{H2} cells also secrete IL-3 and granulocyte-monocyte colony-stimulating factor (GM-CSF).

As used herein, “T_{DTH} refers to the subpopulation of T lymphocytes that initiate delayed hypersensitivity reactions and express, amongst others, the following markers: TCR, CD4, IL-2, and IL-3.

As used herein, T_{CTL} refers to the subpopulation of T lymphocytes that lyse specific target cells. T_{CTL} cells express amongst other markers: TCR and CD8.

As used herein, T_{S} refers to the subpopulation of T cells that suppress immune responses; these cell express amongst other markers: TCR and CD8.

As used herein, “cortical T cells” refers to T cells which express CD7, CD5, CD2, and CD38.

As used herein, “medullary T cells” refers to T cells which express CD1, CD3, CD4, CD8, CD5, CD2, CD7 and CD38.

As used herein, “peripheral T lymphocytes” refers to T lymphocytes which circulate through the blood and other non-thymus lymphoid organs and express either CD4 or CD8.

As used herein, “NK cells” refers to a class of lymphocytes that do not bear markers for either T lymphocytes or B cells, but includes natural killer (NK) cells. “NK cells” express amongst other markers FcR, CD16, perforins and granzymes.

As used herein, “TIL cells” refers to tumor infiltrating lymphocytes extracted from tumor tissue.

As used herein, “LAK cells” refers to lymphokine activated killer cells that do not bear T lymphocyte markers, are not MHC restricted, lyse cell lines resistant to natural killer (NK) cell killing, and are effective in reducing large tumor cell masses in mice. STEWART SELL, 919 (1996).

As used herein, the term “antigen” refers to a substance that elicits an immune response and can include superantigens.

As used herein, the term “sterile inflammatory disease” refers to any inflammatory disease caused by immune or non-immune mechanisms not directly linked to infection (see STEWART SELL ET AL., 1996). Examples of sterile inflammatory diseases include, but are not limited to psoriasis, rheumatoid arthritis, glomerulonephritis, asthma, cardiac and renal reperfusion injury, thrombosis, adult respiratory distress syndrome, inflammatory bowel diseases, such as Crohn's disease and ulcerative colitis and periodontal disease.

As used herein, the term “mitogen” refers to a class of substances that stimulate lymphocytes to proliferate independently of antigen.

As used herein, the term “superantigen” refers to a special class of antigens, defined by their capacity to stimulate a large fraction of T lymphocytes. Superantigens include several “enterotoxins”, which are globular proteins released by such bacteria as Staphylococcus aureus.

As used herein, the term “pathogen” refers to any infectious organism including bacteria, viruses, parasites, mycoplasma, protozoans, and fungi (including molds and yeast). “Pathogenic bacteria” include, but are not limited to Staphylococci (e.g., aureus), Streptococci (e.g., pneumoniae), Clostridia (e.g., perfringens), Neisseria (e.g., gonorrhoeae), Enterobacteriaceae (e.g., E. coli as well as Klebsiella, Salmonella, Shigella, Yersinia and Proteus), Helicobacter (e.g., pylori), Vibrio (e.g., cholerae), Campylobacter (e.g., jejuni), Pseudomonas (e.g., aeruginosa), Haemophilus (e.g., influenzae), Bordetella (e.g., pertussis), Mycoplasma (e.g., pneumoniae), Ureaplasma (e.g., urealyticum), Legionella (e.g., pneumophila), Spirochetes (e.g., Treponema, Leptospira and Borrelia), Mycobacteria (e.g., tuberculosis, smegmatis), Actinomyces (e.g., israelii), Nocardia (e.g., asteroides), Chlamydia (e.g., trachomatis), Rickettsia, Coxiella, Ehrilichia, Rochalimaea, Brucella, Yersinia, Fracisella, Mycobacterium leprae, and Pasteurella.

“Pathogenic viruses” include amongst others: human T lymphocyte virus type I and II (HTLV-I and HTLV-II), Epstein-Barr Virus (EBV), group C adenoviruses, herpes simplex virus, cytomegalovirus, poliovirus, rubella, measles, mumps respiratory syncytial virus (RSV), vesicular stomatitis virus (VSV), HIV-1, rabies virus, influenza A, parainfluenza, and lymphocytic choriomeningitis virus.

As used herein, “immunodeficiency diseases” or “immunodeficiency disorders” include both acquired immunodeficiency and primary immunodeficiency disorders. Primary immunodeficiency disorders encompass: antibody deficiency disorders (e.g., sex linked agammaglobulinemia, common variable immunodeficiency, selective IgA deficiency, immunodeficiency with elevated IgM, transient hypogammaglobulinemia of infancy, antibody deficiency with near normal immunoglobulins, and X-linked lymphoproliferative disease); cellular immunodeficiency disorders (e.g., thymic hypoplasia, and Nezelof's syndrome); and severe combined immunodeficiency (SCID) disorders (e.g., autosomal recessive severe combined immunodeficiency disease, X-linked recessive severe combined immunodeficiency disease, defective expression of major histocompatibility complex antigens, and severe combined immunodeficiency with leukopenia).

The phrase “solid support” refers to any support to which nucleic acids can be bound or immobilized, including nitrocellulose, nylon, glass, other solid supports which are positively charged, including nanochannel glass arrays disclosed by Beattie (WO95/1175).

The phrase “specifically hybridizes” refers to nucleic acids which hybridize under highly stringent or moderately stringent conditions to the nucleic acids containing at least one of the sequences identified in the Figures. In referencing the Figures, we mean at all points the Figures and the discussion of the Figures found in the Brief Description of Drawings. Preferably such specifically hybridizing nucleic acids will present a clear and detectable signal, and they may be labeled by various means that are known to those skilled in the art.

The phrase “isolated nucleic acid” refers to nucleic acids that have been separated from contaminant nucleic acids encoding other polypeptides. “Nucleic acids” refers to all forms of DNA and RNA, including cDNA molecules and antisense RNA molecules.

The phrase “sequences contiguous with” refers to sequences which are covalently linked to a given nucleic acid sequence or fragment at either the 5′ or 3′ end through phospho-diester bonds. Such contiguous sequences are contained within a single molecule with a given nucleic acid sequence or fragment, such as a cDNA molecule.

The phrase “gene expression profile”, also referred to as a “differential expression profile” or “expression profile”, refers to a representation of the expression of mRNA species in a cell sample or population. For instance, a gene expression profile can refer to an autoradiograph of labeled cDNA fragments produced from total cellular mRNA separated on the basis of size by known procedures. Such procedures include slab gel electrophoresis, capillary gel electrophoresis, high performance liquid chromatography (HPLC), and the like. Digitized representations of scanned electrophoresis gels are also included as are two and three dimensional representations of the digitized data.

While a gene expression profile encompasses a representation of the expression level of at least one mRNA species, in practice, the typical gene expression profile represents the expression level of multiple mRNA species. For instance, a gene expression profile useful in the methods and compositions disclosed herein represents the expression levels of at least about 1, 5, 10, 20, 50, 100, 150, 200, 300, 500, 1000, 10,000, 50,000 or, more preferably, substantially all of the detectable mRNA species in a cell sample or population. Particularly preferred are gene expression profiles or arrays affixed to a solid support that contain a representative number of mRNA species, whose expression levels are modulated under the relevant infection, disease, screening, treatment or other experimental conditions. In some instances, a sufficient representative number of such mRNA species will be at least about 1, 2, 5, 10, 15, 20, 25, 30, 40, 50, 50-75 or 100. The expression profiles preferably will also be able to quantitatively differentiate the relative quantities of mRNA species in a cell.

Gene expression profiles can be produced by any means known in the art, including, but not limited to the methods disclosed by: Liang et al., (1992) Science 257: 967-971; Ivanova et al., (1995) Nucleic Acids Res. 23: 2954-2958; Guilfoyl et al., (1997) Nucleic Acids Res. 25(9): 1854-1858; Chee et al., (1996) Science 274: 610-614; Velculescu et al., (1995) Science 270: 484-487; Fischer et al., (1995) Proc. Natl. Acad. Sci. USA 92(12): 5331-5335; and Kato, (1995) Nucleic Acids Res. 23(18): 3685-3690. Gene expression profiles also may be produced by the methods of Belyavsky et al., in U.S. patent application Ser. No. 08/499,899. Preferably, gene expression profiles are produced by the methods of Prashar et al., (WO 97/05286) and Prashar et al., (1996) Proc. Natl. Acad. Sci. USA 93: 659-663.

As an example, gene expression profiles as described herein are made to identify one or more genes whose expression levels are modulated in a T lymphocyte population exposed to a pathogen, allergen, antigen, mitogen or superantigen, or a T lymphocyte population isolated, for example, from a subject having a sterile inflammatory disease, GVHD or cancer. The assaying of the modulation of gene expression via the production of a gene expression profile generally involves the production of cDNA from polyA RNA (mRNA) isolated from T lymphocytes as described below.

The mRNAs are isolated from a T lymphocyte source (e.g., prothymocytes or T_{H2} cells) The cells may be obtained from an in vivo source, such as a peripheral blood or thymus. As is apparent to one skilled in the art, any lymphocyte type (e.g., plasma cells, B cells, T lymphocytes or T lymphocyte subpopulations) may be used, however, T lymphocytes are preferred. Furthermore, the peripheral blood cells that are initially obtained may be subjected to various separation techniques (e.g., flow cytometry, density gradients) to purify specific T lymphocyte subpopulations (e.g., T_{DTH}, T_{CTL}, T_{H2}, etc.).

“mRNAs” are isolated from cells by any one of a variety of techniques. Numerous techniques are well known (see e.g., SAMBROOK ET AL., MOLECULAR CLONING: A LABORATORY APPROACH, Cold Spring Harbor Press, NY, (1987); AUSUBEL ET AL., CURRENT PROTOCOLS IN MOLECULAR BIOLOGY, Greene Publishing Co. NY, (1995)). In general, these techniques first lyse the cells and then enrich for or purify RNA. In one such protocol, cells are lysed in a Tris-buffered solution containing sodium dodecyl sulfate. The lysate is extracted with phenol/chloroform, and nucleic acids are precipitated. Purification of poly(A)-containing RNA is not a requirement. The mRNAs may, however, be purified from crude preparations of nucleic acids or from total RNA by chromatography, such as binding and elution from oligo(dT)-cellulose or poly(U)-Sepharose®. As stated above, other protocols and methods for isolation of RNAs may be substituted.

The mRNAs are reverse transcribed using an RNA-directed DNA polymerase, such as “reverse transcriptase” isolated from such retroviruses as AMV, MoMuLV or recombinantly produced. Many commercial sources of enzyme are available (e.g., Pharmacia, New England Biolabs, Stratagene Cloning Systems). Suitable buffers, cofactors, and conditions are well known and supplied by various manufacturers (see also, SAMBROOK ET AL., (1989) supra; AUSUBEL ET AL., (1995) supra).

Various oligonucleotides are used in the production of cDNA. In particular, the methods described herein utilize oligonucleotide primers for cDNA synthesis, and adapters, and primers for amplification. Oligonucleotides are generally synthesized as single strands by standard chemistry techniques, including automated synthesis. Oligonucleotides are subsequently de-protected and may be purified by precipitation with ethanol, chromatographed using a sized or reversed-phase column, denaturing polyacrylamide gel electrophoresis, high-pressure liquid chromatography (HPLC), or other suitable method. In addition, within certain preferred embodiments, a functional group, such as biotin, is incorporated preferably at the 5′ or 3′ terminal nucleotide. A biotinylated oligonucleotide may be synthesized using pre-coupled nucleotides, or alternatively, biotin may be conjugated to the oligonucleotide using standard chemical reactions known to individuals skilled in the art. Other functional groups, such as florescent dyes, radioactive molecules, digoxigenin, and the like, may also be used.

Partially-double stranded adaptors are formed from single-stranded oligonucleotides by annealing complementary single-stranded oligonucleotides that are chemically synthesized or by enzymatic synthesis. Following synthesis of each strand, the two oligonucleotide strands are mixed together in a buffered salt solution (e.g., 1 M NaCl; 100 mM Tris-HCl, pH 8.0; 10 mM EDTA) or in a buffered solution containing Mg⁺² (e.g., 10 mM MgCl₂) and annealed by heating to high temperature and slow cooling to room temperature.

The oligonucleotide primer that primes first strand DNA synthesis comprises a 5′ sequence incapable of hybridizing to a polyA tail of the mRNAs, and a 3′ sequence that hybridizes to a portion of the polyA tail of the mRNAs and at least one non-polyA nucleotide immediately upstream of the polyA tail. The 5′ sequence is preferably of a sufficient length such that can serve as a primer for amplification. The 5′ sequence also preferably has an average G+C content and does not contain large palindromic sequences; certain palindromes, such as a recognition sequence for a restriction enzyme, may be acceptable. Examples of suitable 5′ sequences are CTCTCAAGGATC:TACCGCT (SEQ ID NO.35), CAGGGTAGACGACGCTACGC (SEQ ID NO.36), and TAATACCGCGCCACATAGCA (SEQ ID NO.37).

The 5′ sequence is joined to a 3′ sequence comprising a sequence that hybridizes to a portion of the polyA tail of mRNAs and at least one non-polyA nucleotide immediately upstream. Although the polyA-hybridizing sequence is typically a homopolymer of dT or dU, it need only contain a sufficient number of dT or dU bases to hybridize to polyA under the conditions employed. Both oligo-dT and oligo-dU primers have been used and give comparable results. Thus, other bases may be interspersed or concentrated, as long as hybridization is not impeded. Typically, 12 to 18 bases or 12 to 30 bases of dT or dU will be used. However, as one skilled in the art appreciates, the length need only be sufficient to obtain hybridization. The non-polyA nucleotides are A, C, or G, or a nucleotide derivative, such as inosinate. If one non-polyA nucleotide is used, then three oligonucleotide primers are needed to hybridize to all mRNAs. If two non-polyA nucleotides are used, then 12 primers are needed to hybridize to all mRNAs (AA, AC, AG, AT, CA, CC, CG, CT, GA, GC, GG, GT). If three non-poly A nucleotides are used, then 48 primers are needed (3×4×4). Although there is no theoretical upper limit on the number of non-polyA nucleotides, practical considerations make the use of one or two non-polyA nucleotides preferable.

For cDNA synthesis, the mRNAs are either subdivided into three (if one non-polyA nucleotide is used) or twelve (if two non-polyA nucleotides are used) fractions, each containing a single oligonucleotide primer, or the primers may be pooled and contacted with a mRNA preparation. Other subdivisions may alternatively be used. Briefly, first strand cDNA is initiated from the oligonucleotide primer by reverse transcriptase (RTase). As noted above, RTase may be obtained from numerous sources and protocols are well known. Second strand synthesis may be performed by RTase (Gubler and Hoffman, Gene 25: 263, 1983), which also has a DNA-directed DNA polymerase activity, with or without a specific primer, by DNA polymerase 1 in conjunction with RNaseH and DNA ligase, or other equivalent methods. The double-stranded cDNA is generally treated with phenol:chloroform extraction and ethanol precipitation to remove protein and free nucleotides.

Double-stranded cDNA is subsequently digested with an agent that cleaves in a sequence-specific manner. Such cleaving agents include restriction enzymes. Restriction enzyme digestion is preferred; enzymes that are relatively infrequent cutters (e.g., ≧5 bp recognition site) are more preferred, and those that leave overhanging ends are especially preferred. A restriction enzyme with a six base pair recognition site cuts approximately 8% of cDNAs, so that approximately 12 such restriction enzymes should be needed to digest every cDNA at least once. By using 30 restriction enzymes, digestion of every cDNA is assured.

The adapters for use in the present invention are designed such that the two strands are only partially complementary and only one of the nucleic acid strands that the adapter is ligated to can be amplified. Thus, the adapter is partially double-stranded (i.e., comprising two partially hybridized nucleic acid strands), wherein portions of the two strands are non-complementary to each other and portions of the two strands are complementary to each other. Conceptually, the adapter is “Y-shaped” or “bubble-shaped.” When the 5′ region is non-paired, the 3′ end of other strand cannot be extended by a polymerase to make a complementary copy. The ligated adapter can also be blocked at the 3′ end to eliminate extension during subsequent amplifications. Blocking groups include dideoxynucleotides or any other agent capable of blocking the 3′-OH. In this type of adapter (“Y-shaped”), the non-complementary portion of the upper strand of the adapters is preferably a length that can serve as a primer for amplification. As noted above, the non-complementary portion of the lower strand need only be one base, however, a longer sequence is preferable (e.g., 3 to 20 bases; 3 to 15 bases; 5 to 15 bases; or 14 to 24 bases). The complementary portion of the adapter should be long enough to form a duplex under conditions of litigation.

For “bubble-shaped” adapters, the non-complementary portion of the upper strands is preferably a length that can serve as a primer for amplification. Thus, this portion is preferably 15 to 30 bases. Alternatively, the adapter can have a structure similar to the Y-shaped adapter, but has a 3′ end that contains a moiety that a DNA polymerase cannot extend from.

Amplification primers are also used in the present invention. Two different amplification steps are performed in the preferred aspect. In the first, the 3′ end (referenced to mRNA) of double stranded cDNA that has been cleaved and ligated with an adapter is amplified. For this amplification, either a single primer or a primer pair is used. The sequence of the single primer comprises at least a portion of the 5′ sequence of the oligonucleotide primer used for first strand cDNA synthesis. The portion need only be long enough to serve as an amplification primer. The primer pair consists of a first primer whose sequence comprises at least a portion of the 5′ sequence of the oligonucleotide primer as described above; and a second primer whose sequence comprises at least a portion of the sequence of one strand of the adapter in the non-complementary portion. The primer will generally contain all the sequence of the non-complementary potion, but may contain less of the sequence, especially when the non-complementary portion is very long, or more of the sequence, especially when the non-complementary portion is very short. In some embodiments, the primer will contain a sequence of the complementary portion, as long as that sequence does not appreciably hybridize to the other strand of the adapter under the amplification conditions employed. For example, in one embodiment, the primer sequence comprises four bases of the complementary region to yield a 19 base primer, and amplification cycles are performed at 56° C. (annealing temperature), 72° C. (extension temperature), and 94° C. (denaturation temperature). In another embodiment, the primer is 25 bases long and has 10 bases of sequence in the complementary portion. Amplification cycles for this primer are performed at 68° C. (annealing and extension temperature) and 94° C. (denaturation temperature). By using these longer primers, the specificity of priming is increased.

The design of the amplification primers will generally follow well-known guidelines, such as average G-C content, absence of hairpin structures, inability to form primer-dimers and the like. At times, however, it will be recognized to those skilled in the art that deviations from such guidelines may be appropriate or desirable.

After amplification, the lengths of the amplified fragments are determined. Any procedure that separate nucleic acids on the basis of size and allows detection or identification of the nucleic acids is acceptable. Such procedures include slab gel electrophoresis, capillary gel electrophoresis, high performance liquid chromatography (HPLC), and the like.

Electrophoresis is technique based on the mobility of DNA in an electric field. Negatively charged DNA migrates towards a positive electrode at a rate dependent on its total charge, size, and shape. Most often, DNA is electrophoresed in agarose or polyacrylamide gels. For maximal resolution, polyacrylamide is preferred and for maximal linearity, a denaturant, such as urea is present. A typical get setup uses a 19:1 mixture of acrylamide:bisacrylamide and a Tris-borate buffer. DNA samples are denatured and applied to the gel, which is usually sandwiched between glass plates. A typical procedure can be found in SAMBROOK ET AL., (1989) or AUSUBEL ET AL., (1995). Variations may be substituted as long as sufficient resolution is obtained.

Capillary electrophoresis, (CE) in its various manifestations (e.g., free solution, isotachophoresis, isoelectric focusing, polyacrylamide gel, micellar electrokinetic “chromatography”), allows high resolution separation of very small sample volumes. Briefly, in capillary electrophoresis, a neutral coated capillary, such as a 50 μm×37 cm column (eCAP neutral, Beckman Instruments, CA), is filled with a linear polyacrylamide (e.g., 0.2% polyacrylamide), a sample is introduced by high-pressure injection followed by an injection of running buffer (e.g., 1×TBE). The sample is electrophoresed and fragments are detected. An order of magnitude increase can be achieved with the use of capillary electrophoresis. Capillaries may be used in parallel for increased throughput (Smith et al., (1990) Nuc. Acids. Res. 18: 4417; Mathies and Huang, (1992) Nature 359: 167). Because of the small sample volume that can be loaded onto a capillary, the sample may be concentrated to increase the level of detection. One means of concentration is sample stacking (Chien and Burgi, (1992) Anal. Chem. 64: 489A). In sample stacking, a large volume of sample in a low concentration buffer is introduced to the capillary column. The capillary is then filled with a buffer of the same composition, but at higher concentration, such that when the sample ions reach the capillary buffer with a lower electric field, they stack into a concentrated zone. Sample stacking can increase detection by one to three orders of magnitude. Other methods of concentration, such as isotachophoresis, may also be used.

High-performance liquid chromatography (HPLC) is a chromatographic separation technique that separates compounds in solution. HPLC instruments consist of a reservoir of mobile phase, a pump, an injector, a separation column, and a detector. Compounds are separated by injecting an aliquot of the sample mixture onto the column. The different components in the mixture pass through the column at different rates due to differences in their partitioning behavior between the mobile liquid phase and the stationary phase. IP-RO-HPLC on non-porous PS/DVB particles with chemically bonded alkyl chains can also be used to analyze nucleic acid molecules on the basis of size (Huber et al., (1993) Anal. Biochem. 121: 351; Huber et al., (1993) Nuc. Acids Res. 21: 1061; Huber et al., (1993) Biotechniques 16: 898).

In each of these analysis techniques, the amplified fragments are detected. A variety of labels can be used to assist in detection. Such labels include, but are not limited to, radioactive molecules (e.g., ³⁵S, ³²P, ³³P), fluorescent molecules, and mass spectrometric tags. The labels may be attached to the oligonucleotide primers or to nucleotides that are incorporated during DNA synthesis, including amplification.

Radioactive nucleotides may be obtained from commercial sources; radioactive primers may be readily generated by transfer of label from γ-³²P-ATP to a 5′-OH group by a kinase (e.g., T4 polynucleotide kinase). Detection systems include autoradiograph, phosphor image analysis and the like.

Fluorescent nucleotides may be obtained from commercial sources (e.g., ABI, Foster City, Calif.) or generated by chemical reaction using appropriately derivatized dyes. Oligonucleotide primers can be labeled, for example, using succinimidyl esters to conjugate to amine-modified oligonucleotides. A variety of florescent dyes may be used, including 6 carboxyfluorescein, other carboxyfluorescein derivatives, carboxyrhodamine derivatives, Texas red derivatives, and the like. Detection systems include photomultiplier tubes with appropriate wave-length filters for the dyes used. DNA sequence analysis systems, such as produced by ABI (Foster City, Calif.), may also be used.

After separation of the amplified cDNA fragments, cDNA fragments which correspond to differentially expressed mRNA species are isolated, reamplified and sequenced according to standard procedures. For instance, bands corresponding the cDNA fragments can be cut from the electrophoresis gel, reamplified and subcloned into any available vector, including pCRscript using the PCR script cloning kit (Stratagene). The insert is then sequenced using standard procedures, such as cycle sequencing on an ABI sequencer.

An additional means of analysis comprises hybridization of the amplified fragments to one or more sets of oligonucleotides or nucleic acid fragments immobilized on a solid substrate. Historically, the solid substrate is a membrane, such as nitrocellulose or nylon. More recently, the substrate is a silicon wafer or a borosilicate slide. The substrate may be porous (Beattie et al., WO 95/11755) or solid. Oligonucleotides are synthesized in situ or synthesized prior to deposition on the substrate. Various chemistries are known for attaching oligonucleotide. Many of these attachment chemistries rely upon functionalizing oligonucleotides to contain a primary amine group. The oligonucleotides are arranged in an array form, such that the position of each oligonucleotide sequence can be determined.

The amplified fragments, which are generally labeled according to one of the methods described herein, are denatured and applied to the oligonucleotides on the substrate under appropriate salt and temperature conditions. In certain embodiments, the conditions are chosen to favor hybridization of exact complementary matches and disfavor hybridization of mismatches. Unhybridized nucleic acids are washed off and the hybridized molecules detected, generally both for position and quantity. The detection method will depend upon the label used. Radioactive labels, fluorescent labels and mass spectrometry label are among the suitable labels.

The present invention as set forth in the specific embodiments, includes methods to identify a therapeutic agent that modulates the expression of at least one gene in a T lymphocyte population. Genes which are differentially expressed during T lymphocyte contact with a pathogen, antigen, superantigen, mitogen or allergen, or that are differentially expressed in a subject having a sterile inflammatory disease, cancer or GVHD are of particular importance.

In general, the method to identify a therapeutic or prophylactic agent that modulates the response of a T lymphocyte population to a pathogen, mitogen, antigen, superantigen or allergen, comprises the steps of preparing a first gene expression profile of a quiescent T lymphocyte population or subpopulation, preparing a second gene expression profile of a T lymphocyte population or subpopulation exposed to a pathogen, mitogen, antigen, superantigen or allergen, treating the exposed T lymphocyte population or subpopulation with the agent, preparing a third gene expression profile of the treated T lymphocyte population, comparing the first, second and third gene expression profiles and identifying agents that modulate the response of a T lymphocyte population or subpopulation to the pathogen, mitogen, antigen, superantigen or allergen.

In another format, the method is used to identify a therapeutic agent that modulates the expression of genes in a T lymphocyte population or subpopulation found in a human or animal subject having a sterile inflammatory disease, GVHD or cancer. The general method comprises the steps of preparing a first gene expression profile of a T lymphocyte population or subpopulation in a subject having a sterile inflammatory disease GVHD or cancer, treating or exposing the T lymphocyte population or subpopulation to the agent, preparing a second gene expression profile of the treated T lymphocyte population or subpopulation, comparing the first and second gene expression profiles with the gene expression profile of a normal T lymphocyte population and identifying agents that modulate the expression of genes whose transcription levels are altered in the T lymphocyte population or subpopulation of the subject as compared with normal T lymphocyte population or subpopulation.

While the above methods for identifying a therapeutic agent comprise the comparison of gene expression profiles from treated and not-treated T lymphocytes, many other variations are immediately envisioned by one of ordinary skill in the art. As an example, as a variation of a method to identify a therapeutic or prophylactic agent that modulates the response of a T lymphocyte population or subpopulation to pathogen, mitogen, antigen, superantigen or allergen, the second gene expression profile of a T lymphocyte population or subpopulation exposed to a pathogen, antigen, mitogen, immunogen or allergen and the third gene expression profile of the treated T lymphocyte population or subpopulation can each be independently normalized using the first gene expression profile prepared from a quiescent T lymphocyte population or subpopulation. Normalization of the profiles can easily be achieved by scanning autoradiographs corresponding to each profile, and subtracting the digitized values corresponding to each band on the autoradiograph from quiescent T lymphocytes from the digitized value for each corresponding band on autoradiographs corresponding to the second and third gene expression profiles. After normalization, the second and third gene expression profiled can be compared directly to detect cDNA fragments which correspond to mRNA species, which are differentially expressed upon exposure of the T lymphocyte population or subpopulation to the agent to be tested.

Nucleic Acid Fragments

Nucleic acids of the claimed invention include nucleic acids which specifically hybridize to nucleic acids comprising the sequences identified in the Figures. A nucleic acid which specifically hybridizes to a nucleic acid comprising one of the sequences identified in the Figures remains stably bound to said nucleic acid under highly stringent or moderately stringent conditions. Stringent and moderately stringent conditions are those commonly defined and available, such as those defined by Sambrook et al., (1989) or Ausubel et al., (1995). The precise level of stringency is not important, rather, conditions should be selected that provide a clear, detectable signal when specific hybridization has occurred.

Hybridization is a function of sequence identity (homology), G+C content of the sequence, buffer salt content, sequence length and duplex melt temperature (Tₘ) among other variables. See, Maniatis et al., (Molecular Cloning, Cold Spring Harbor Laboratory, Cold Spring Harbor, N.Y. 1982). With similar sequence lengths, the buffer salt concentration and temperature provide useful variables for assessing sequence identity (homology) by hybridization techniques. For example, where there is at least 90 percent homology, hybridization is commonly carried out at 68° C. in a buffer salt such as 6×SCC diluted from 20×SSC. See Sambrook et al., (1989) The buffer salt utilized for final Southern blot washes can be used at a low concentration, e.g., 0.1×SSC and at a relatively high temperature, e.g., 68° C., and two sequences will form a hybrid duplex (hybridize). Use of the above hybridization and washing conditions together are defined as conditions of high stringency or highly stringent conditions. Moderately stringent conditions can be utilized for hybridization where two sequences share at least about 80 percent homology. Here, hybridization is carried out using 6×SSC at a temperature of about 50-55° C. A final wash salt concentration of about 1-3×SSC and at a temperature of about 60-68° C. are used. These hybridization and washing conditions define moderately stringent conditions.

In particular, specific hybridization refers to conditions in which a high degree of complementarity exists between a nucleic acid comprising the sequences identified in at least one of the Figures and another nucleic acid. With specific hybridization, complementarity will generally be at least about 75%, 80%, 85%, preferably about 90-100%, or most preferably about 95-100%.

The nucleic acids of the present invention can be used in a variety of ways in accordance with the present invention. For example, they can be used as nucleic acid probes to screen other cDNA and genomic DNA libraries so as to select by hybridization other DNA sequences that comprise similar sequences. The nucleic acid probe could be RNA or DNA labeled with radioactive nucleotides or by non-radioactive methods (i.e., biotin). Screening could be done at various stringencies (through manipulation of the hybridization Tₘ, usually using a combination of ionic strength, temperature and/or presence of formamide) to isolate close or distantly related homologs. The nucleic acids may also be used to generate primers to amplify cDNA or genomic DNA using polymerase chain reaction (PCR) techniques. The nucleic acid sequences of the present invention can also be used to identify adjacent sequences in the cDNA or genome, for example, flanking sequences and regulatory elements.

The nucleic acid sequences of the present invention can also be used diagnostically to detect nucleic acid sequences which specifically hybridize to at least one of the sequences identified in the Figures. For instance, said sequences can be used to detect activated T lymphocytes or T lymphocytes previously exposed to a specific pathogen, mitogen, antigen, superantigen or allergen. As set forth in the Examples, said sequences are the partial sequences of cDNA species which correspond to T lymphocyte mRNA and therefore genes, which are differentially expressed during T lymphocyte contact with a specific pathogen, mitogen, antigen, superantigen or allergen. Nucleic acid fragments comprising at least part of these sequences may be used as diagnostic probes to identify T lymphocyte populations that have been activated or have been in contact with a specific pathogen, mitogen, antigen, superantigen or allergen.

In general, nucleic acid fragments comprising at least one of the sequences or part of one of the sequences identified in the Figures can be used as probes to screen nucleic acid samples from T lymphocyte populations in hybridization assays. Such assays can be used to detect activated T lymphocytes or T lymphocytes exposed to a specific pathogen, mitogen, antigen, superantigen or allergen. To ensure specificity of a hybridization assay using a probe derived from such sequences, it is preferable to design probes which hybridize only with target nucleic acid under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes may be designed from the sequences of the Figures through methods known in the art. For instance, the G+C content of the probe and the probe length can affect probe binding to its target sequence. Methods to optimize probe specificity are commonly available in Sambrook et al., (1989), or Ausubel et al., (1995).

The nucleic acid sequences of the present invention can also be used as probes to monitor the expression of at least one differentially expressed T lymphocyte gene in a method to identify a therapeutic or prophylactic agent that modulates the response of a T lymphocyte population to a specific pathogen, mitogen, antigen, superantigen or allergen. In general, the method to identify a therapeutic or prophylactic agent that modulates the response of a T lymphocyte population to a specific pathogen, mitogen, antigen, superantigen or allergen comprises the steps of determining the expression level of at least one RNA species that specifically hybridizes to a probe comprising all or part of at least of one of the sequences identified in the Figures in a quiescent T lymphocyte population. The expression level of the RNA species is then determined in a T lymphocyte population exposed to a specific pathogen, mitogen, antigen, superantigen or allergen and also in a T lymphocyte population exposed to a specific pathogen, mitogen, antigen, superantigen or allergen and to the agent to be tested. Agents which modulate the expression level of a RNA species associated with T lymphocyte activation by a specific pathogen, mitogen, antigen, superantigen or allergen are thereby identified by comparing the expression levels of the RNA species.

Hybridization assays to determine the expression level of at least one RNA species are commonly available and include the detection of DNA:RNA and RNA:RNA hybrids. Northern blots of total cellular RNA or polyA purified RNA and hybridization assays wherein at least one or part of one of the sequences of the present invention are immobilized to a solid support are included.

Solid supports can be prepared that comprise immobilized representative groupings of nucleic acids corresponding to the sequences or parts of the sequences identified in the Figures. For instance, representative nucleic acids can be immobilized to any solid support to which nucleic acids can be immobilized, such as positively charged nitrocellulose or nylon membranes (see Sambrook et al., 1989), as well as porous glass wafers such as those disclosed by Beattie (WO 95/11755). Nucleic acids are immobilized to the solid support by well established techniques, including charge interactions, as well as attachment of derivatized nucleic acids to silicon dioxide surfaces such as glass which bears a terminal epoxide moiety. A solid support comprising a representative grouping of nucleic acids can then be used in standard hybridization assays to detect the presence or quantity of one or more specific nucleic acid species in a sample (such as a total cellular mRNA sample or cDNA prepared from said mRNA) which hybridize to the nucleic acids attached to the solid support. Any hybridization methods, reactions, conditions and/or detection means can be used, such as those disclosed by Sambrook et al., (1989), Ausbel et al., (1987), or Beattie (WO 95/11755).

One of ordinary skill in the art may determine the optimal number of nucleic acid species that must be represented by nucleic acid fragments immobilized on the solid support to effectively differentiate between samples, e.g., T lymphocytes exposed to a specific pathogen, mitogen, antigen, superantigen or allergen. Preferably, at least about 1, 5, 10, 20, 28 or more nucleic acid fragments corresponding to at least one or part of one of the sequences identified in the Figures are affixed to a solid support. The skilled artisan will be able to optimize the number and particular nucleic acids for a given purpose, i.e., screening for modulating agents, identifying activated T lymphocytes, etc.

Full Length cDNA Fragments

The determination of the sequences identified in the Figures as derived from T lymphocyte mRNA species (genes) that are differentially regulated in response to exposure of a T lymphocyte population to a specific pathogen, mitogen, antigen, superantigen or allergen enables the isolation of full length cDNA molecules encoding proteins associated with the T lymphocyte response.

As set forth above, any method may be used to prepare a cDNA library from T lymphocytes. Preferably, the cDNA library is produced from T lymphocytes exposed to a specific pathogen, mitogen, antigen, superantigen or allergen. After exposure, the cDNA library is prepared by extracting the mRNA from isolated T lymphocytes, using known methods, for example, isolation of polyadenylated (poly A+) RNA. Kits for isolating poly A+ RNA are commercially available, for example, PolyATract kits are available from Promega Corporation. The mRNA thus extracted may be enriched for mRNAs corresponding to genes differentially expressed by hybrid selection procedures, and the like. In instances of a low recovery of RNA, mRNA can be preamplified by PCR using known methods. The cDNAs corresponding to the mRNAs may be prepared using a reverse transcriptase for first strand synthesis and a DNA polymerase for second strand synthesis. Methods for using reverse transcriptase and DNA polymerase to make cDNA are well known in the art. Kits for performing these techniques are commercially available, for example, the Superscript II™ kit (Gibco-BRL), the Great Lengths cDNA Synthesis Kit™ (Clontech), the cDNA Synthesis Kit (Stratagene), and the like.

The cDNAs may then be ligated to linker DNA sequences containing suitable restriction enzyme recognition sites. Such linker DNAs are commercially available, for example, from Promega Corporation and from New England Biolabs and the particular linker used may be selected to conform to the protocol being used. The cDNAs may be subjected to restriction enzyme digestion, size fractionation, or any other suitable method, to enrich for full-length cDNAs within the library.

The resultant cDNA library can be modified to select for rare transcripts or for transcripts corresponding to genes that are differentially expressed upon exposure to a specific pathogen, mitogen, antigen, superantigen or allergen. For instance, when using a sequence corresponding to a mRNA which is up-regulated in response to exposure of a T lymphocyte population to a specific pathogen, mitogen, antigen, superantigen or allergen, a cDNA library is prepared from exposed T lymphocytes and subtractively hybridized to cDNA or mRNA (polyA+ RNA) from a quiescent T lymphocyte population. Subtractive hybridization methods are available, for instance as taught by Davis et al., (1987) Cell 51: 987-1000; Hedrick et al., (1984) Nature, 308: 149-153; and Sargent et al., (1983) Science, 222: 135-139. Subtractive library methods that utilize PCR amplification may also be used as taught, for example, by Wieland et al., (1990) Proc. Natl. Acad. Sci. USA, 87: 2720-2724; Wang et al., (1991) Proc. Natl. Acad. Sci. USA, 88: 11505-11509; Cecchini et al., (1993) Nucleic Acids Res., 21: 5742-5747; Lebeau et al., (1991) Nucleic Acids Res., 19: 4778; Duguid et al., (1990) Nucleic Acids Res., 18: 2789-2792. and U.S. Pat. No. 5,525,471

The resultant cDNA library may also be normalized to obtain cDNAs corresponding to rare or weakly expressed mRNA species. Many procedures are available including hybridization of the cDNA library to genomic DNA as taught by Weissman et al., (1987) Mol. Biol. Med., 4: 133-143. Other available techniques include utilizing second order hybridization kinetics to select for rarer species as taught by Ko et al., (1990) Nuc. Acids. Res., 18: 5709; Patanjali et al., (1991) Proc. Natl. Acad. Sci. USA, 88: 1943-1947 or Soares et al., (U.S. Pat. No. 5,637,685).

The cDNA library is then screened from cDNA clones that specifically hybridize to a nucleic acid comprising at least one or part of one of the sequences identified in the Figures. Such methods are widely available as set forth above.

After isolation of cDNA clones which specifically hybridize to a nucleic acid comprising at least one or part of one of the sequences identified in the Figures, the inserts into the cDNA molecules can be further characterized by known methods including the sequencing of the cDNA insert. In instances where the 5′ end of the cDNA encompassing the amino terminus of an encoded protein is not contained within a cDNA molecule isolated by the above methods, 5′RACE PCR amplification and other known procedures can be used to retrieve the 5′ end of the cDNA. Such methods are known in the art and exemplified by Fang et al., (1997) Biotechniques, 23 (1): 52, 54, 56, 58; Chen (1996) Trends Genet., 12 (3): 87-88; Lung et al., (1996) Trends Genet., 12 (10): 389-91; Bahring et al., (1994) Biotechniques, 16 (5): 807-8; and Borson et al., (1992) PCR Methods Appl., 2(2): 144-8.

Related Nucleic Acids

As used herein, the present invention encompasses nucleic acids wherein sequences flanking or contiguous with at least one of the sequences identified in the Figures include: sequences remaining in the same open reading frame; sequences which do not include a stop codon; sequences which terminate at a stop codon; sequences which serve as a promoter, operator or other regulatory control sequence; or sequences which are derived from genomic DNA.

Vectors and Host Cells

The present invention comprises recombinant vectors containing and capable of replicating and directing the expression of nucleic acids comprising at least one, or part of one of the sequences identified in the Figures in a compatible host cell. The insertion of a DNA in accordance with the present invention into a vector may be performed by any conventional means. Such an insertion is easily accomplished when both the DNA and the desired vector have been cut with the same restriction enzyme or enzymes, since complementary DNA termini are thereby produced. If this cannot be accomplished, it may be necessary to modify the cut ends that are produced by digesting back single-stranded DNA to produce blunt ends, or by achieving the same result by filling in the single-stranded termini with an appropriate DNA polymerase. In this way, blunt-end ligation with an enzyme such as T4 DNA ligase may be carried out. Alternatively, any site desired may be produced by ligating nucleotide sequences (linkers) onto the DNA termini. Such linkers may comprise specific oligonucleotide sequences that encode restriction site recognition sequences.

Any available vectors and the appropriate compatible host cells may be used such as those disclosed by Sambrook et al., (1989) and Ausubel et al., (1995). Commercially available vectors, for instance, those available from New England Biolabs Inc., Promega Corp., Stratagene Inc. or other commercial sources are included.

### SPECIFIC EMBODIMENTS

### Example 1

### Production of Gene Expression Profiles Generated from cDNAs Made with RNA Isolated from Quiescent T Lymphocytes (Jurkat Lymphocytes) and Activated T Lymphocytes

Expression profiles of RNA expression levels from Jurkat lymphocytes exposed to activating agents, such as 12-O-tetradecanoylphorbol-13-acetate (TPA) and phytohemagglutinin (PHA), offer a powerful means of identifying genes that are specifically regulated during T lymphocyte activation.

Culturing of Jurkat Lymphocytes and Treatment with an Activating Agent

Jurkat lymphocytes (Jurkat clone E6-1; ATCC Accession No. TIB 152) were grown in 6×100 ml RPMI culture media supplemented with 10% fetal bovine serum (FBS), penicillin, streptomycin and glutamine. The Jurkat lymphocytes were pelleted in 450 ml RPMI media supplemented with penicillin, streptomycin and glutamine only and were incubated for approximately 24 hours. After 24 hours, FBS was added to a final concentration of 10%. The flasks of Jurkat lymphocytes were incubated for 1 hour. Cells were then pelleted, resuspended in 100 ml PBS, pelleted again and resuspended ultimately in 500 ml RPMI supplemented with 10% FBS, glutamine, streptomycin and penicillin. A small aliquot of the cells was removed, stained with trypan blue and counted. The concentration of cells was determined to be 2.45×10⁶ (or 1.225×10⁹ total cells). From the 500 ml suspension, 250 ml was removed to another flask and 250 ml fresh 10% FBS RPMI was added to both flasks making equal aliquots of 500 ml containing ˜1.0×10⁶ cells each. For the non-stimulated (resting or quiescent) Jurkat lymphocytes, five 100 ml flasks were made from one of the 500 ml flasks. The remaining 500 ml flask of Jurkat lymphocytes was treated (stimulated) with 500 μl of 2.0 mg/ml phytohemagglutinin (PHA) and 25 μl of 1.0 mg/ml tetraphorbol acetate (TPA), which was dissolved in ethanol. Jurkat lymphocytes could also be stimulated using ionomycin (Sigma) and TPA (or phorbol derivatives). The 500 ml of stimulated cells were then aliquoted into five 100 ml tissue culture flasks. Cells were harvested 4 hours after the addition of PHA and TPA. The cells were pelleted at 1.5×g for at least 5 minutes and the supernatant was removed by pouring. Pellets were then placed on ice for the RNA to be extracted.

cDNA Preparation

Total cellular RNA was prepared from the untreated and treated Jurkat lymphocytes using the procedure of Newburger et al., (1981) J. Biol. Chem. 266(24): 16171-7 and Newburger et al., (1988) Proc. Natl. Acad. Sci. USA 85: 5215-5219. Ten micrograms of total RNA, the amount obtainable from about 3×10⁶ Jurkat lymphocytes, is sufficient for a complete set of cDNA expression profiles.

Synthesis of cDNA was performed as previously described by Prashar et al., in WO 97/05286 and in Prashar et al., (1996) Proc. Natl. Acad. Sci. USA 93: 659-663. Briefly, cDNA was synthesized according to the protocol described in the GIBCO/BRL kit for cDNA synthesis. The reaction mixture for first-strand synthesis included 6 μg of total RNA, and 200 ng of a mixture of 1-base anchored oligo(dT) primers with all three possible anchored bases as shown in the table for FIG. 1: (ACGTAATACGACTCACTATAGGGCGAATTGGGTCGACTTTTTTTTTTTTTT TTTn1 wherein n1=A/C or G) (SEQ ID NO.38) along with other components for first-strand synthesis reaction, except reverse transcriptase. This mixture was incubated at 65° C. for 5 min, chilled on ice and the process repeated. Alternatively, the reaction mixture may include 10 μg of total RNA, and 2 pmol of one of the 2-base anchored oligo(dT) primers as a heel such as RP5.0 (CTCTCAAGGATCTTACCGCTT₁₈AT) (SEQ ID NO.39), or RP6.0 (TAATACCGCGCCACATAGCAT₁₈CG) (SEQ ID NO.40), or RP9.2 (CAGGGTAGACGACGCTACGCT₁₈GA) (SEQ ID NO. 41) along with other components for first-strand synthesis reaction except reverse transcriptase. This mixture was then layered with mineral oil and incubated at 65° C. for 7 min followed by 50° C. for another 7 min. At this stage, 2 μl of Superscript reverse transcriptase (200 units/μl; GIBCO/BRL) was added quickly and mixed, and the reaction continued for 1 hr at 45-50° C. Second-strand synthesis was performed at 16° C. for 2 hr. At the end of the reaction, the cDNAs were precipitated with ethanol and the yield of cDNA was calculated.

The adapter oligonucleotide sequences were A1 (TAGCGTCCGGCGCAGCGACGGCCAG) (SEQ ID NO.42) and A2 (GATCCTGGCCGTCGGCTGTCTGTCGGCGC) (SEQ ID NO.43). One microgram of oligonucleotide A2 was first phosphorylated at the 5′ end using T4 polynucleotide kinase (PNK). After phosphorylation, the oligonucleotide was heat denatured, and 1 μg of the oligonucleotide A1 was added along with 10× annealing buffer (1 M NaCl; 100 mM Tris-HCl, pH 8.0; 10 mM EDTA, pH 8.0) in a final vol of 20 μl. This mixture was then heated at 65° C. for 10 min followed by slow cooling to room temperature for 30 min, resulting in formation of the Y adapter at a final concentration of 100 ng/μl. About 20 ng of the cDNA was digested with 4 units of Bgl II or another restriction enzyme in a final vol of 10 μl for 30 min at 37° C. Two μl (≈4 ng of digested cDNA) of this reaction mixture was then used for ligation to 100 ng (≈50-fold) of the Y-shaped adapter in a final vol of 5 μl for 16 hr at 15° C. After ligation, the reaction mixture was diluted with water to a final vol of 80 μl (adapter ligated cDNA concentration, 50 pg/μl), heated at 65° C. for 10 min to denature T4 DNA ligase, and 2 μl aliquots (with 100 pg of cDNA) were used for PCR.

The following sets of primers were used for PCR amplification of the adapter ligated 3′-end cDNAs:

TGAAGCCGAGACGTCGGTCG(T)₁₈ n1, n2 (wherein n1, n2=AA, AC, AG AT CA CC CG CT GA GC GG or GT) (SEQ ID NO. 44) as the 3′ primer with A1 as the 5′ primer or alternatively, opposing primers used as 3′ primers with primer A1.1 serving as the 5′ primer. To detect the PCR products on the display gel, 24 pmol of oligonucleotide A1 or A1.1 was 5′-end-labeled using 15 μl of [γ-³²P]-ATP (Amersham; 3,000 Ci/mmol) and PNK in a final volume of 20 μl for 30 min at 37° C. After heat denaturing at 65° C. for 20 min, the labeled oligonucleotide was diluted to a final concentration of 2 μM in 80 μl with unlabeled oligonucleotide A1.1. The PCR mixture (20 μl) consisted of 2 μl (100 pg) of the template, 2 μl of 10×PCR buffer (100 mM Tris.HCl, pH 8.3; 500 mM KCl), 2 μl of 15 mM MgCl₂ to yield 1.5 mM final Mg²⁺ concentration optimum in the reaction mixture, 200 μM dNTPs, 200 nM each 5′ and 3′ PCR primers (RP primers), and 1 unit of Amplitaq Gold. Primers and dNTPs were added after preheating the reaction mixture containing the rest of the components at 85° C. This “hot start” PCR was done to avoid amplification of artifacts arising out of arbitrary annealing of PCR primers at lower temperature during transition from room temperature to 94° C. in the first PCR cycle. PCR consisted of 5 cycles of 94° C. for 30 sec; 55° C. for 2 min, and 72° C. for 60 sec, followed by 25 cycles of 94° C. for 30 sec, 60° C. for 2 min, and 72° C. for 60 sec. A higher number of cycles resulted in smeary gel patterns. PCR products (2.5 μl) were analyzed on 6% polyacrylamide sequencing gel. For double or multiple digestion following adapter ligation, 13.2 μl of the ligated cDNA sample was digested with a secondary restriction enzyme(s) in a final vol of 20 μl. From this solution, 3 μl was used as template for PCR. This template volume of 3 μl carried 100 pg of the cDNA and 10 mM MgCl₂ (from the 10× enzyme buffer), which diluted to the optimum of 1.5 mM in the final PCR volume of 20 μl. Since Mg²⁺ comes from the restriction enzyme buffer, it was not included in the reaction mixture when amplifying secondarily cut cDNA.

Production of Gene Expression Profiles Generated Using all 12 Possible Anchoring Oligo d(T) n1, n2.

Using the above described methods, RNA was extracted and the cDNA profiles prepared using all 12 possible anchoring oligo d(T)n1, n2.

FIG. 1A is an autoradiogram of the expression profiles generated from cDNAs made with RNA isolated from control (untreated) Jurkat lymphocytes and Jurkat lymphocytes incubated with TPA and PHA.

FIG. 1B the expression profiles generated from cDNAs made with RNA isolated from control (untreated) Jurkat lymphocytes and activated (treated) Jurkat lymphocytes is an autoradiogram of activated and quiescent Jurkat lymphocytes using the RP 8.6 primers.

Such autoradiography gels may be scanned using commonly available equipment, such as a UMAX D-1L scanner. Bands which exhibit altered intensities in gene expression profiles from quiescent Jurkat lymphocytes as compared to the gene expression profile prepared from activated (treated) Jurkat lymphocytes can then be extracted from the display gel as previously described above. The isolated fragments can then be reamplified using 5′ and 3′ primers, subcloned into pCR-Script (Stratagene) and sequenced using an ABI automated sequencer. Alternatively, bands can be extracted (cored) from the display gels, PCR amplified and sequenced directly without subcloning.

### Example 2

### Production of Gene Expression Profiles Generated from cDNAs Made with RNA Isolated from T Lymphocytes Exposed to Staphylococci or Streptococci

Expression profiles from T lymphocytes (Jurkat lymphocytes) exposed to virulent and avirulent Staphylococcus aureus and Streptococcus would allow the identification of T lymphocyte genes that are specifically regulated in response to bacterial infection by these organisms, as well as in response to the superantigens, the bacterialenterotoxins, they contain.

T lymphocytes are cultured as described above in Example 1. The cells are then exposed to either Staphylococci or Streptococci or the enterotoxins therefrom. When exposing the T lymphocytes to an enterotoxin, the T cells can be directly exposed by the addition of enterotoxin to the culture medium or by addition of bacterial cells to the culture medium. Before incubation, bacteria are harvested and washed in phosphate buffered saline. T lymphocytes are then incubated with the bacteria at a ratio which induces activation of the T lymphocytes. For instance, a T lymphocyte:bacteria ratio of 1:20 would be appropriate in RPMI 1640 (HEPES buffered) in the presence of heat inactivated FBS at 37° C. with gentle mixing in a rotary shaker bath.

In instances where an antigen specific response is required, bacterial antigens may be presented to the T lymphocytes in the context of class I or class II major histocompatibility antigens (MHC). Many methods to present antigen in the context of MHC are readily available, including the exogenous loading of bacterial antigens onto antigen presenting cells, including macrophages and dendritic cells. Such methods include treating an antigen-presenting cell to enhance expression by the cell of empty major histocompatibility complex molecules, followed by reacting the treated antigen presenting cell with an antigen extracorporeally in the presence of a photoactivatable agent and irradiation to form an antigen-associated antigen presenting cell. (See, e.g., PCT application number US93/11220, publication number WO 94/11016). Other available methods include the production of antigen fusions to such molecules as the Bordetella pertussis adenylate cyclase (see U.S. Pat. No. 5,679,784), lysosomal/endosomal targeting sequences (see U.S. Pat. No. 5,633,234), the production of empty class II heterodimers comprising antigenic peptides (see U.S. Pat. No. 5,583,031), or the use of exogenously loaded immortalized dendritic cells (see U.S. Pat. No. 5,648,219).

After non-specific activation by exotoxin or antigen specific activation by a Staphylococcal or Streptococcal antigen, expression profiles from the activated cells as well as a quiescent T lymphocyte control population may be made by any means available in the art. Preferably, gene expression profiles are generated as described in Example 1. After autoradiography or another means of detection, cDNA bands corresponding to mRNA species which are differentially expressed are visualized and the band(s) isolated or excised for further analysis as in Example 1.

### Example 3

### Production of Gene Expression Profiles Generated from cDNAs Made with RNA Isolated from Human T Lymphocytes Exposed to Ionomycin and sn-1,2-dioctanoylglycerol

Human T lymphocytes were isolated using the method of Subramaniam et al., (1988) Cell. Immunol. 116: 439-449. The cells were treated with ionomycin and sn-1,2-dioctanoylglycerol (diC8) in the same media conditions as described in Example 1. The concentrations of ionomycine and diC8 used to activated the human T lymphocytes were as described in Subramaniam et al.

After incubation, RNA was extracted from the treated and untreated human T lymphocytes, and gene expression profiles prepared as described in Example 1. FIG. 2A is an autoradiogram of the expression profiles generated from cDNAs made with RNA isolated from control (untreated) Jurkat lymphocytes and human T lymphocytes incubated with ionomycin and diC8.

As exhibited by FIGS. 2A-2B, the expression of mRNA species in human T lymphocytes exposed to ionomycin and diC8 (as indicated by their corresponding cDNA fragments) is modulated compared to the expression of mRNA species of unexposed (quiescent) human T lymphocytes. cDNA bands corresponding to mRNA species which are differentially expressed upon exposure to ionomycin and diC8 are identified by comparing the gene expression profile for the treated T lymphocytes to the gene expression profile from the untreated control T lymphocytes and the band(s) isolated or excised for further analysis as in Example 1.

### Example 4

### Production of Gene Expression Profiles Generated from cDNAs Made with RNA Isolated from Activated Human T Lymphocytes Exposed to Cyclosporin

The ability to compare gene expression profiles from activated T lymphocytes to gene expression profiles prepared from activated T lymphocytes treated or exposed to cyclosporin or another immunoinhibitory agent allows the identification and isolation of mRNA species, and therefore genes, that are differentially expressed upon the deactivation of a T lymphocyte population.

Human peripheral blood quiescent T lymphocytes are acquired according to the procedures set forth in Sehajpal et al., Cell. Immunol. 120: 195-204 (1989) and Subramaniam et al., (1988). An aliquot of the T lymphocyte population is activated using diC8 and ionomycin, as described in Example 3. A second aliquot of the T lymphocyte population is treated with diC8, ionomycin and cyclosporin A (CsA). Gene expression profiles are then prepared from the diC8 and ionomycin treated cells, the diC8, ionomycin and cyclosporin A treated cells and a control untreated quiescent T lymphocyte population according to the methods set forth in Example 1. The resulting gene expression profiles are then compared to identify cDNA bands corresponding the mRNA species that are differentially regulated upon exposure to cyclosporin. For instance, cDNA species corresponding to mRNA species that are either up-regulated or down-regulated in an activated T lymphocyte population upon exposure to cyclosporin are isolated or excised for further analysis, as in Example 1.

### Example 5

### Method to Identify a Therapeutic or Prophylactic Agent that Modulates the Response of a Proliferating T Lymphocyte Population

The methods set forth in Examples 1 and 3 offer a powerful approach for identifying therapeutic or prophylactic agents that modulate the activity of T lymphocytes or specific T lymphocyte subpopulations (e.g., T_{H1} or T_{H2}) to either antigen-specific or antigen-nonspecific activation. For instance, an aliquot of a T lymphocyte population that has been activated via an antigen specific or antigen-nonspecific pathway is exposed to the agent to be tested. Gene expression profiles are then prepared as set forth in Example 1. A gene expression profile is also prepared from a control aliquot of the activated T lymphocyte population that has not been exposed to the agent to be tested as well as a quiescent T lymphocyte population. By examining for differences in the intensity of individual bands between the three gene expression profiles, agents which up or down regulate the expression of one or more mRNA species in activated T lymphocytes are identified.

As a specific example, screening for agents which down regulate the expression of at least one gene associated with T lymphocyte activation, such as the human serine esterase gene, provides a means for identifying agents which may be useful as immunoinhibitory drugs. As set forth in the Table of FIG. 4, a cDNA band corresponding to the human serine esterase mRNA is among a number of cDNA bands that are up-regulated upon T lymphocyte activation. To screen for such agents, an aliquot of a T lymphocyte population that has been activated via an antigen specific or antigen-nonspecific pathway is exposed to the agent to be tested. Gene expression profiles are then prepared as set forth in Example 1. Gene expression profiles are also prepared from a control aliquot of the activated T lymphocyte population that has not been exposed to the agent to be tested as well as a quiescent T lymphocyte population. By examining for differences in the intensity of individual cDNA bands identified as being up-regulated in the Table S of FIG. 4 or 5, such as the cDNA band corresponding the mRNA encoding human serine esterase, agents which down regulate the expression of one or more mRNA species in activated T lymphocytes are identified. Agents that down regulate expression of human serine esterase, as demonstrated by decreased band density in the profile produced from activated T lymphocytes exposed to the agent, may be useful in modulating the activation of a T lymphocyte population.

### Example 6

### Method to Identify a Therapeutic or Prophylactic Agent that Modulates the Response of a T Lymphocyte Population to a Pathogen

The methods set forth in Examples 1 and 3 also offer a powerful approach for identifying therapeutic or prophylactic agents that modulate the activation of T lymphocytes or specific T lymphocyte subpopulations (e.g., T_{H1} or T_{H2}) to a pathogen (e.g., a virus, bacteria or fungi) or parasite. An aliquot of an isolated T lymphocyte population or subpopulation is exposed to a pathogen or parasite of interest such as Staphylococcus aureus, a Streptococcus species or Mycobacterium leprae. Exposure of the T lymphocyte population to the pathogen or parasite antigens can be facilitated by the presentation of pathogen or parasite antigens to the T lymphocyte population in the context of class I or class II MHC using commonly available methods, such as those disclosed in Example 2. A second aliquot of the same T lymphocyte population is then exposed to the pathogen or parasite antigens as above in the presence of the agent to be tested. Gene expression profiles are then prepared from the two aliquots, as well as from a quiescent T lymphocyte population, by the methods set forth in Example 1. By examining for differences in the intensity of individual bands between the three profiles, agents which up- or down-regulate genes of interest in the pathogen or parasite exposed T lymphocytes can be identified. Such agents can be used, for example, as immunostimulatory agents to up-regulate antigen specific T lymphocyte activation.

### Example 7

### Method to Identify a Therapeutic or Prophylactic Agent that Modulates the Activity of a T Lymphocyte Population Found in a Subject Having a Sterile Inflammatory Disease, Immunodeficiency Disorder, Autoimmune Disorder or T Lymphocyte Neoplasm

The methods set forth in Examples 1 and 3 also offer a powerful approach for identifying therapeutic or prophylactic agents that modulate the expression of T lymphocytes or T lymphocyte subpopulations in subjects exhibiting the symptoms of a sterile (non-infectious) inflammatory disease, immunodeficiency disorder, autoimmune disorder or T lymphocyte neoplasm (hereinafter in this example to be referred to as “T lymphocyte disease”). T lymphocytes from a subject exhibiting the symptoms of a T lymphocyte disease are isolated according to readily available methods, for instance, the methods disclosed in Subramaniam et al., (1988) Cell. Immunol. 116: 439-449.

To test agents for their effects on T gene expression, an aliquot of the T lymphocyte population isolated from the subject is then treated or exposed to the agent to be tested. Gene expression profiles are then prepared from the aliquot of the T cell population exposed to the agent, from an aliquot of the same T lymphocyte population isolated from the subject and from a quiescent T lymphocyte population isolated from a normal subject not exhibiting the symptoms of a T lymphocyte disease the according to the methods set forth in Example 1. By examining these profiles for differences in the intensity of bands between the three profiles, agents which up- or down-regulate genes of interest in a T lymphocyte population from a subject exhibiting the symptoms of a T lymphocyte disease can be identified. Agents that up-regulate a gene or genes that are expressed at abnormally low levels in a T lymphocyte population from a subject exhibiting the symptoms of a T lymphocyte disease compared to a normal T lymphocyte population, as well as agents that down-regulate a gene or genes that are expressed at abnormally high levels in a T lymphocyte population from a subject exhibiting the symptoms of a T lymphocyte disease are identified.

### Example 8

### Method of Isolating Agents that Modulate Virus Infected T Lymphocytes

The methods set forth in Examples 1 and 3 are also useful for identifying therapeutic or prophylactic agents that modulate the response of T lymphocytes or T lymphocyte subpopulations to viral infection. Many viruses demonstrate a tropism for T lymphocyte populations, including but not limited to HIV and HTLV I.

T lymphocyte populations infected with a given virus are isolated from a subject exhibiting symptoms of viral infection. Alternatively, normal uninfected T lymphocyte cell lines or uninfected T lymphocytes are isolated from an uninfected subject and infected with the virus of interest in vitro. An aliquot of the T lymphocyte population infected with the virus is then exposed to the agent to be tested. Expression profiles are then prepared from the treated T virus infected T lymphocyte aliquot, an aliquot of the virally infected T lymphocyte population which has not been exposed to the agent, and a control population of normal quiescent T lymphocytes using the methods described in Examples 1 and 3. The profiles are then compared to identify agents that beneficially modulate the T lymphocyte response to viral infection. For instance, agents are identified that modulate the expression level of at least one gene whose expression level is down-regulated upon viral infection. Alternatively, agents are identified that modulate the expression level of at least one gene whose expression level is up-regulated in response to viral infection.

### Example 9

### Method of Isolating Agents that Induce Differentiation of Pre-T Lymphocytes and Cortical or Medullary Thymocytes

The methods set forth in Examples 1 and 3 may be used to identify therapeutic or prophylactic agents that induce differentiation of pre-T lymphocytes and cortical or medullary thymocytes. Pre-T lymphocytes or thymocytes are obtained using flow cytometry and antibodies for specific markers that are expressed on these cells. Once the pre-T lymphocytes or thymocytes are isolated, an aliquot of the cells are treated or exposed to the agent to be tested. An expression profile is then prepared from this exposed cell population, an aliquot of the same cell population which has not been exposed to the agent and a population of differentiated T lymphocytes. This population may be selected from the group consisting of cells of the following lineages: T_{H2}, T_{DTH}, T_{CTL}, T_{H2}, T_{S}, memory T lymphocytes, effector T lymphocytes, pre-T lymphocytes, cortical T lymphocytes, medullary T lymphocytes, and peripheral T lymphocytes. The profiles generated from the exposed T lymphocyte population and the control cells are then compared to the expression profiles of the specific differentiated T lymphocyte subpopulations (either activated or resting T lymphocyte subpopulations) to identify agents that dedicate the pre-T lymphocyte or thymocyte to a particular T lymphocyte subpopulation (e.g., T_{H2}, T_{DTH}, T_{CTL}, T_{H2}, T_{S}, memory T lymphocytes, effector T lymphocytes, pre-T lymphocytes, cortical T lymphocytes, medullary T lymphocytes, and peripheral T lymphocytes).

### Example 10

### Production of Articles to which are Attached or Adsorbed Selected Groupings of Nucleic Acids that Correspond to the Genes Whose Expression Levels are Modulated in a T Lymphocyte Population that has been Exposed to a Pathogen, Mitogen, Immunogen, Allergen, Antigen, or Superantigen

As set forth in Examples 1 and 3, expression profiles from T lymphocytes, for example, that have been exposed to a pathogen, mitogen, immunogen, allergen, antigen or superantigen yield the identity of genes whose expression levels are modulated compared to unexposed T lymphocyte populations.

Suitable solid supports can be prepared by those skilled in the art that comprise immobilized representative groupings of nucleic acids corresponding to the genes or fragments of the genes from T lymphocytes whose expression levels are modulated in response to exposure to a pathogen, mitogen, immunogen, superantigen or antigen. For instance, representative nucleic acids can be immobilized to any solid support to which nucleic acids can be immobilized, such as positively charged nitrocellulose or nylon membranes (see SAMBROOK ET AL., (1989) as well as porous glass wafers, such as those disclosed by Beattie (WO 95/11755). Nucleic acids are immobilized to the solid support by well established techniques, including charge interactions, as well as attachment of derivatized nucleic acids to silicon dioxide surfaces such as glass which bears a terminal epoxide moiety. A solid support comprising a representative grouping of nucleic acids can then be used in standard hybridization assays to detect the presence or quantity of one or more specific nucleic acid species in a sample (such as a total cellular mRNA sample or cDNA prepared from said mRNA), which hybridize to the nucleic acids attached to the solid support. Any hybridization methods, reactions, conditions and/or detection means can be used, such as those disclosed by SAMBROOK ET AL., (1989), AUSBEL ET AL., (1987), or Beattie (WO 95/11755).

One of ordinary skill in the art may determine the optimal number of genes and species of genes should be represented by nucleic acid fragments immobilized on the solid support to effectively differentiate between the gene expression profiles of, e.g., T lymphocytes exposed to various pathogens and T cells not exposed to the pathogens. Preferably, at least about 1, 5, 10, 20, 50, 100, 150, 200, 300, 500, 1000, 10,000, or more preferably, substantially all of the detectable mRNA species in a cell sample or population will be present in the gene expression profile or array affixed to a solid support. More preferably, such profiles or arrays will contain a sufficient representative number of mRNA species whose expression levels are modulated under the relevant infection or other exposure. In most instances, a sufficient representative number of such mRNA species will be at least about 1, 2, 5, 10, 15, 20, 25, 30, 40, 50, 50-75 or 100 in number and will be represented by the nucleic acid molecules or fragments of nucleic acid molecules immobilized on the solid support. The skilled artisan will be able to optimize the number and particular nucleic acids for a given purpose, i.e., screening for modulating agents, identifying activated T lymphocytes and activated T lymphocyte subpopulations, etc.

### Example 11

### Production of Solid Support Articles Comprising Groupings of Nucleic Acids that Correspond to the Genes Whose Expression Levels are Modulated in a T Lymphocyte Population from a Subject Having a Sterile Inflammatory Disease, Autoimmune Disorder, Immunodeficiency Disorder or T Lymphocyte Neoplasm

The method, compositions and solid support articles set forth in Example 10 can also be used to prepare a solid support that presents nucleic acids that correspond to genes whose expression levels are modulated in T lymphocytes from a subject having a T lymphocyte disease compared to normal, quiescent T lymphocytes, to prothymocytes, or to various subpopulations of T lymphocytes. Solid supports may also be prepared that comprise immobilized representative groupings of nucleic acids corresponding to the genes or fragments of said genes from T lymphocytes whose expression levels are modulated in the subject.

### Example 12

### Method of Diagnosing Exposure of a Subject to a Pathogen

Expression profiles of RNA expression levels from T lymphocytes exposed to various pathogens or pathogen antigens offer a powerful means to diagnose exposure of a subject to a pathogen. As set forth in Examples 1 and 3, the display patterns generated from cDNAs made with RNA isolated from T lymphocytes exposed to pathogenic S. aureus or Streptococcus may exhibit unique patterns of cDNA species corresponding to T lymphocyte mRNA species (genes) whose expression levels are modulated in response to contact of the T lymphocytes with the bacteria or with the bacterial enterotoxin. The contacting of T lymphocytes with different species of pathogens or pathogen antigen may result in the production of expression profiles that are unique to each pathogen species or strain. These unique expression profiles are useful in diagnosing whether a subject has been exposed to or is infected with a given pathogen.

Briefly, expression profiles are produced as set forth in Example 1 and 3, using T lymphocyte samples exposed to various pathogens, such as pathogenic strains of Staphylococci or Streptococci. T lymphocytes are then isolated from the subject to be tested for exposure to a pathogen and an expression profile prepared from the subject's T lymphocytes by the methods set forth above. The expression profile prepared from the subject T lymphocytes can then be compared to the expression profiles prepared from T lymphocytes exposed to, for example pathogenic Staphylococci or Streptococci, and also compared to expression profiles of cells exposed to non-pathogenic strains or quiescent lymphocytes. From such a comparison, it can be determined which expression profile most closely matches the expression profile prepared from the subject, thereby, diagnosing exposure of the subject to a pathogen.

### Example 13

### Method of Diagnosing a Sterile Inflammatory Disease, Autoimmune Disorders, or Immunodeficiency Disorders in a Subject

Expression profiles of RNA expression levels from T lymphocytes isolated from a subject having a sterile inflammatory disease, autoimmune or immunodeficiency disorder offer a powerful means to diagnose inflammatory diseases (e.g., psoriasis, rheumatoid arthritis, glomerulonephritis, asthma, allergic rhinitis, cardiac and renal reperfusion injury, thrombosis, adult respiratory distress syndrome, inflammatory bowel diseases such as Crohn's disease and ulcerative colitis, periodontal disease, etc.). As set forth in Examples 1 and 3, the gene expression profiles generated from cDNAs made with RNA isolated from T lymphocytes from subjects having various sterile inflammatory diseases, autoimmune disorders or immunodeficiency disorders may exhibit unique patterns of cDNA species corresponding to T lymphocyte mRNA species (genes), whose expression levels are modulated during the inflammatory process. These unique expression profiles are useful in diagnosing whether a subject has a sterile inflammatory disease.

Briefly, expression profiles are produced as previously set forth, using T lymphocyte samples isolated from patients with various sterile inflammatory diseases. T lymphocytes are then isolated from the patient to be tested (e.g., diagnosed) and an expression profile prepared from the patient's T lymphocytes by the methods previously set forth. The expression profile prepared from the subject T lymphocytes can then be compared to the expression profiles prepared from T lymphocytes isolated from patients with various sterile inflammatory diseases, immunodeficiency disorders, or autoimmune disorders to determine which expression profile most closely matches the expression profile prepared from the patient, thereby, diagnosing whether the patient has a sterile inflammatory disease, immunodeficiency disorder or autoimmune disorder.

### Example 14

### Method of Diagnosing Graft Versus Host Disease (GVHD) in a Subject

Expression profiles of mRNA expression levels from T lymphocytes isolated from a subject suffering from or possibly suffering from graft versus host disease (GVHD) offer a powerful means of diagnosing GVHD prior to the expression of severe symptoms. The early detection of GVHD could aid in decreasing the morbidity associated with allografts and xenografts.

Briefly, expression profiles are obtained from an individual who received an autologous graft or less preferably, from a normal person. An expression profile of T lymphocytes obtained from individuals at various stages in the course of GVHD would also be obtained. The two expression profiles would then be compared to the expression profile obtained from a patient who recently received an allograft or xenograft. From such a comparison, it can be determined whether the patient is rejecting his or her grafted tissue. Such early detection of GVHD may save the life of the transplant recipient.

It should be understood that the foregoing discussion and examples merely present a detailed description of certain preferred embodiments. It therefore should be apparent to those of ordinary skill in the art that various modifications and equivalents can be made without departing from the spirit and scope of the invention. All references, articles and patents identified above are herein incorporated by reference in their entirety.

1. A method to identify a therapeutic or prophylactic agent that modulates that response of a lymphocyte population to an antigen, comprising the steps of:
preparing a first gene expression profile of a quiescent lymphocyte population;
preparing a second gene expression profile of a lymphocyte population exposed to the antigen;
treating the exposed lymphocyte population with a candidate compound;
preparing a third gene expression profile of the treated lymphocyte population;
comparing the first, second and third gene expression profiles; and
identifying as a therapeutic of prophylactic agent a compound that modulates the response of a lymphocyte population to the antigen.

2. The method of claim 1, wherein the lymphocyte is a T lymphocyte.

3. (canceled)

4. The method of claim 2, wherein the T lymphocyte population is selected from the group consisting of T_{H1}, T_{DTH}, T_{CTL}, T_{H2}, T_{S}, memory T lymphocytes, effector T lymphocytes, pre-T lymphocytes, cortical T lymphocytes, medullary T lymphocytes, peripheral T lymphocytes, activated T lymphocytes, quiescent T lymphocytes, and neoplastic T lymphocytes,

5. The method of claim 2, wherein the antigen is selected from the group consisting of a pathogen, an antigen derived from a pathogen, an allergen, a superantigen or self-antigen.

6. The method of claim 5, wherein the pathogen is selected from the group consisting of bacteria, viruses, parasites, mycoplasma, protozoans, and fungi.

7. The method of claim 6, wherein the virus is selected from the group consisting of EBV, HIV-1, HTLV-1, HTLV-II, rabies virus, mouse mammary tumor virus, cytomegalovirus, poliovirus, Group C adenoviruses, herpes simplex virus, rubella, measles, mumps, respiratory syncytial virus, vesicular stomatitis virus, influenza A, parainfluenza, and lymphocytic choriomeningitis virus.

8. The method of claim 2, wherein the T lymphocyte population is exposed to the antigen associated with major histocompatibility molecules.

9. The method of claim 8, wherein the major histocompatibility molecules are exposed on the surface of antigen presenting cells.

10. The method of claim 8, wherein the major histocompatibility molecules are selected from the group consisting of Class I MHC and Class II MHC.

11. The method of claim 9, wherein the antigen presenting cells are selected from the group consisting of B lymphocytes, macrophages and dendritic cells.

12. A method to identify a therapeutic or prophylactic agent that modulates a T lymphocyte population found in a subject having a sterile inflammatory disease, autoimmune disorder, immunodeficiency disease, cancer, or GVHD comprising the steps of:
preparing a first gene expression profile of a T lymphocyte population in a subject having the sterile inflammatory disease, autoimmune disorder, immunodeficiency disease, cancer, or GVHD;
treating the T lymphocyte population with a candidate compound;
preparing a second gene expression profile of the treated T lymphocyte population;
comprising the first and second gene expression profiles with a gene expression profile of a normal T lymphocyte population; and
identifying as a therapeutic of prophylactic agent a compound that modulates a T lymphocyte population found in a subject having a sterile inflammatory disease, autoimmune disorder, immunodeficiency disease, cancer, or GVHD.

13-20. (canceled)

21. A method of diagnosing a sterile inflammatory disease, autoimmune disorder, immunodeficiency disease, cancer, or GVHD in a subject, comprising the steps of:
preparing a first gene expression profile of a T lymphocyte population from the subject;
comparing the first gene expression profile to at least one second gene expression profile from a T lymphocyte population from a subject having a sterile inflammatory disease, autoimmune disorder, immunodeficiency disease, cancer, or GVHD and to a third gene expression profile of a normal T lymphocyte population; and
determining if the subject has a sterile inflammatory disease, autoimmune disorder, immunodeficency disease, cancer, or GVHD.

22-33. (canceled)


SEQUENCE LISTING (44 sequences)

SEQ ID NO: 1 - DNA, 238 bp, Homo sapiens
  unsure (11), (30), (32), (231): n= a or c or g or t; Jurkat cell cDNA
gatcctatgtnccccccaggcggctggcantncccacgggaagtgtccactgaggtccctgagatggatacctctacctgacatggcctgaagatgcagggcagaggaattgcccatggacagtgacgcaaggactaggctgggagggagcgtgccaaccccttttgcctctgggtttggggagcggagggcctcttcttggtgccctgcccccaataaaggaactggacnaagagat

SEQ ID NO: 2 - DNA, 174 bp, Homo sapiens
  unsure (150): n = a or c or g or t; Jurkat cell cDNA
gatctcatgatgtggctgttgggaagatggtggggtttgtttgccagcttggagtcctattaaatgaaagccagcaactcatgttggtaataggtctactgtgggaacagttatccctaaccacagctcaaaatcgctatcatctttagncaaattaaaatctatgtggcagcg

SEQ ID NO: 3 - DNA, 175 bp, Homo sapiens
  Jurkat cell cDNA
gatctggtgactggcttttcgttctgtgttcttggcttcctaaatttatctgcccatatgattctcatgcatttgatatttatgtttaaaagtgtttatatatgtatgtaaaaagggaaccatatgttttgagaatttgtaaagtgagagacatgatcctattaaaataagaagg

SEQ ID NO: 4 - DNA, 285 bp, Homo sapiens
  unsure (39): n = a or c or g or t; Jurkat cell cDNA
gatcctccatggcccagcaaggcccaagataaatccttnaccacccaggcaccctgtgagcccaacaggttaattagtccattaattttagtgggacctgcatatgttgaaaattaccaatactgactgacatgtgatgctgacctatgataaggttgagtatttattagatgggaagggaaatttggggattatttatcctcctggggacagtttggggaggattatttattgtatttatattgaattatgtacttttttcaataaagtcttatttttgtggcg

SEQ ID NO: 5 - DNA, 182 bp, Homo sapiens
  unsure (33), (56), (75), (93), (105), (110), (122), (136): Jurkat cell cDNA; n = a or c or g or t
gatctgaaacccaggttaggcatgacatttcanccccaaaccctacctcatctgtnctgaaagacgctgaaactncctgggatgttttcgggnacaagaatgtanatttnccctatccctgnacttggtttaatcnaatcaatgtgtgtattagaataaaagtcacagcatcccaaaagccg

SEQ ID NO: 6 - DNA, 130 bp, Homo sapiens
  Jurkat cell cDNA
caggatcttaaaaatcccagccatctaaatatgtttcccaactccattaagtaaggtaaaataatatttgtatttatgttcagatgttgaagctgtcattctcgaataaaactacactttagaaatggcg

SEQ ID NO: 7 - DNA, 361 bp, Homo sapiens
  unsure (62), (80): n = a or c or g or t; Jurkat cell cDNA
gatctttcgaggccaggtgcccaggtctttcatcaagagccccatttccaagtgctcagtanccccttttggccagtgcncccccaccacatgggacaagcgcaggtccagtggcctccccagctgaccgcaggcagggaacaaggcagaccctagagggccaggccacagcaggggctgaggatgcctggtgaatggatgcctgggagaatggatgccagaattcacgcatgaggctctgaacagggctgggaaaacttccaaacgaagggaagctcatgtcttggtgcactttgtgatgatgcttcaacagcaggactgagatggggacatttacaataaacagaaatgtatgggctcg

SEQ ID NO: 8 - DNA, 176 bp, Homo sapiens
  Jurkat cell cDNA
ggatcttgcacgtatctgttttcctcccccatgaactagaaaaccacttactcccagaattcaggtcgtgcttgttagtactatatcaccaagtccattcatttaatgatccaaaactgtaatgttgcactgtattccaaataaagggtaaaaacagaaccaaagttataactccg

SEQ ID NO: 9 - DNA, 128 bp, Homo sapiens
  unsure (57), (58): n = a or c or g or t; Jurkat cell cDNA
gatcaattctatgtctgactttgaaattccatttacaatgtagtatgttttcaatgnnaaaccataaagtaacatccaagtgtttcatggtttgttgggaaggtaattttaaaataaaacaatttccg

SEQ ID NO: 10 - DNA, 138 bp, Homo sapiens
  Jurkat cell cDNA
gatcaagtcactgcatgttgagaagtataggtataacttgtgaccatatcacagctcctttatttatgtagtttcttcacattttatgtgtacaatcaagcatgcctgctgaccaaggccagaggtggagtggaagcg

SEQ ID NO: 11 - DNA, 271 bp, Homo sapiens
  Jurkat cell cDNA
gatctcaacattgttggtttcttttgtttttcatttggtacaactttcttgaatttagaaattacatctttgcagttctgttaggtgctctgtaattaacctgacttatatgtgaacaattttcatgagacagtcatttttaactaatgaagtgattctttctcactactatctgtattgtggaatgcacaaaattgtgtaggtgctgaatgctgtaaggagtttaggttgtatgaattctacaaccctataataaattttactctatacg

SEQ ID NO: 12 - DNA, 186 bp, Homo sapiens
  unsure (20), (115): n = a or c or g or t; Jurkat cell cDNA
gatccaaaactatttggganatgtatgggtagggtaaatcagtaagaggtgttatttggaaccttgttttggacagtttaccagttgccttttatcccaaagttgttgtaacctnctgtgatacgatgcttcaagagaaaatgcggttataaaaaatggttcagaattaaacttctaattcattcg

SEQ ID NO: 13 - DNA, 171 bp, Homo sapiens
  Jurkat cell cDNA
ggatctgacctccacggagccgctgtccccgcccccctgctcccgtctgtctgtcctgtctgattctcttaggtgtcatgttcttttttctgtcttgtcttcaactttttttaaaactagattgctttgaaaacatgactcaataaaagtttcctttcaatttaaacaccg

SEQ ID NO: 14 - DNA, 151 bp, Homo sapiens
  T lymphocyte cDNA
agctccagaagcgcttggacaggctggaggagacagtccaggccaagtagagccccacagggcctccagcagggtcagccattcacacccatccactcacctcccattcccagccacgtggcagagaaaaaaatcataataaaatggcttt

SEQ ID NO: 15 - DNA, 148 bp, Homo sapiens
  unsure (3), (4), (13), (30), (40): n = a or c or g or t; T lymphocyte cDNA
ttnngctacctgngtccaagtcttggcttnccctttccantcacttcactgtgcgctaaggggtggggtgaggggatggagagggagggctgcctaccatggtctggggcttgaggaagatgagtttgttgatttaaataaagaattt

SEQ ID NO: 16 - DNA, 194 bp, Homo sapiens
  unsure (4), (19), (21): n = a or c or g or t; T lymphocyte cDNA
ctantttagatacgtccananccaggaccgctgagaactgggacagtttcctgggatgagtgccagcctgagcctgcatggtgccgccgagcccggggtggaggagggagccaggcttcgcttcaaggcggcctctaccttttctcagaatggtttcctgattgtgtcaatgtgaaagttaaataaaatttatg

SEQ ID NO: 17 - DNA, 116 bp, Homo sapiens
  unsure (7)..(9), (18), (22), (24), (28), (29), (31), (33),(42), (44),(46), (47), (52), (55), (58), (63), (69), (75),(78), (79): n = a or c or g or t; T lymphocyte cDNA
cactgtnnngaacggtcntgcnangtannangncttctgccnangnntctcnctncanccaanaggcanctttcntanntatcctaacaagccttggaccaaatggaaataacagc

SEQ ID NO: 18 - DNA, 212 bp, Homo sapiens
  T lymphocyte cDNA
gctttattggagagatacacacaaaggctgtccactcacttccataatttcttgatggacatgtttttctcactgtccttctgcatgaccttggctactgccatctcaaagtcctcctgagtgacatggactcgccgttctcgcagggcatacatgccagcttctgtgcacacgcccttcacttcaagcccctgatgctcctggcatgagct

SEQ ID NO: 19 - DNA, 189 bp, Homo sapiens
  T lymphocyte cDNA
tgcatttatggaaggcacattacaggtctttgtgggaagaaacagaaagaaatcacaaaagcaattaagagagctcaaataatggggtttatgccagttacatacaaggatcctgcatatctcaaggaccctaaagtttgtaacatcagatatcgggaataaattctatcacgttaccactaataaact

SEQ ID NO: 20 - DNA, 219 bp, Homo sapiens
  unsure (2), (5): n = a or c or g or t; T lymphocyte cDNA
antgnagggaaagctatgaaaggtgccggcggatctacaacatggaaatggctcgcaagatcaacttcttgatgcgaaagaatcgggcagatccgtggcagggctgctgaggcctgtgggtgggacacccagtgcgaaaccctcatccagttttctctccatctcttttctttgtacaatcccatttcctattaccattctctgcaataaactcaaatc

SEQ ID NO: 21 - DNA, 285 bp, Homo sapiens
  T lymphocyte cDNA
agctcctccctctggtggtgcttcctcagggcccaccattgaagaggttgattaagccaaccaagtgtagatgtagcattgttccacacatttaaaacatttgaaggacctaaattcgtagcaaattctgtggcagttttaaaaagttaagctgctatagtaagttactgggcattctcaatacttgaatatggaacatatgcacaggggaaggaaataacattgcactttataaacactgtattgtaagtggaaaatgcaatgtcttaaataaaactatttaaa

SEQ ID NO: 22 - DNA, 195 bp, Homo sapiens
  unsure (4), (12): n = a or c or g or t; T lymphocyte cDNA
ctantttagatncgtccacagccaggaccgctgagaactgggacagtttcctgggatgagtgccagcctgagcctgcatggtgccgccgagcccggggtggaggagggagccaggcttcgcttcaaggcggcctctaccttttctcagaatggtttcctgattgtgtcaatgtgaaagttaaataaaatttatgt

SEQ ID NO: 23 - DNA, 180 bp, Homo sapiens
  T lymphocyte cDNA
tttgtgccgtctctccattccactgcctgttgcagagtttttctgtaactaagggggttgaggttattgtagacgttagattgcgggcaccgccagggattttgcagcgcttcagtgtacgtgttagagaatattggaaaagcgtctgtgagccccgtgctgtattttgtaataaagtct

SEQ ID NO: 24 - DNA, 138 bp, Homo sapiens
  unsure (4): n = a or c or g or t; T lymphocyte cDNA
aggntctctgagcacttaccgggcgtgaccgtttcttaggtgtgagaggggctgtggcttttgtgcagcgactatgttggtgttaggggtggtgtggagattgttaatcttgtataaagcaattcaataaattgtttc

SEQ ID NO: 25 - DNA, 74 bp, Homo sapiens
  unsure (5), (6), (11): n = a or c or g or t; T lymphocyte cDNA
cgagnngcaancttctgaggcggtgtgtgcacaagcctttcagggggcacattcacaagtacctgttgtgtccc

SEQ ID NO: 26 - DNA, 119 bp, Homo sapiens
  unsure (4), (8), (11), (17), (25): n = a or c or g or t; T lymphocyte cDNA
tgtntccntgnaagggnccttgcanagtaatagggcttctgcctaagcctctccctccaagccaataggcagctttcttaactatcctaacaagccttggaccaaatggaaataaagct

SEQ ID NO: 27 - DNA, 253 bp, Homo sapiens
  unsure (4), (5), (15), (20), (32), (33), (37), (42), (45),(47),(50), (52): n = a or c or g or t; T lymphocyte cDNA
gtgnnccagtcttgncttgnccaccgcccagnnacangctgntcngnatnantatgaagagctcaatgtctggcaggtcaatgcttcccggacacggatcacttttgtctgattccagcctgcttgcaaccctggggtcctcttgttccctgctggcctgccccttgggaaggggcagtgatgcctttgaggggaaggaggagcccctctttctcccatgctgcacttactccttttgctaataaaagtgttt

SEQ ID NO: 28 - DNA, 344 bp, Homo sapiens
  unsure (15), (19), (24), (38), (40), (50), (59), (63), (69),(70), (87), (129): n = a or c or g or t; T lymphocyte cDNA
cgagtgtagcacaancatncgacnggcgacttcgccantntcatcctttntgggaacancaanatacannctccatttctggagtcngggtcttccgaagccaggagcttgcctttccgctgagtccanattggcaggtggactacgagtcatacacatggcggaaactggatcctggcagtgaggagacccagacgctggttcgagagtacttttcctgggagggggccttccagcatgtgggcaaagccttcaatcagggcaagatcttcaagtgaacatctcttgccatcacctagctgcctgcacctgcccttcagggagatgggggtcattaaaggaaa

SEQ ID NO: 29 - DNA, 456 bp, Homo sapiens
  unsure (6), (28), (33), (40)..(42), (97), (113), (170),(202), (311),(347), (401), (409), (440), (444), (453), (454): n = a or c or g or t; T lymphocyte cDNA
agtgtntgcccagggctctgatgtgtcnctcanagcttgnnnagcctgacacagctgtcttgtgagggactgagatgcaggatttcttcacgcctcncctttgtgacttcaanagcctctggcatctctttctgcaaaggcacctgaatgtgtctgcgtccctgttagcntaatgtgaggaggtggagagacagcccacccntgtgtccactgtgacccctgttcccatgctgacctgtgtttcctccccagtcatctttcttgttccagagaggtggggctggatgtctccatctctgtctcaactttangtgcactgagctgcaacttcttacttccctactganaataagaatctgaatatacatttgttttcccaaatatttggcatgaaaaggttntggataanttaataagccattcccgggattttgggaaancaanttttacctnnga

SEQ ID NO: 30 - DNA, 122 bp, Homo sapiens
  unsure (6), (18), (20), (27), (45), (53), (77), (78), (83): n = a or c or g or t; T lymphocyte cDNA
cgtggngctcaagtcttnanctgcccnacgggatcaaacctttcnggcctgtnatgattctgaccatttgacttganncacangtgaatctttctcctggtgactcaaataaaagtataatt

SEQ ID NO: 31 - DNA, 320 bp, Homo sapiens
  unsure (4), (6): n = a or c or g or t; T lymphocyte cDNA
aggnanagtccatggggctgccaacttcagacgaacagaagaaacaggagattctgaagaagttcatggatcaacatccggagatggatttttccaaggctaaattcaactagcccctgttttttcctccctgaactcttggggctgagctgcaaccacccaactttctttcccactcttctctgggacttgtgggcctcagggcttggggcaggcatgggactggcccaggcacacaggtcccggggcatcaggagaaaggctgggtcttgggaccttgtcctccccagttggcctactgttacacattaaaacgattt

SEQ ID NO: 32 - DNA, 116 bp, Homo sapiens
  unsure (22), (23), (35), (36), (47): n = a or c or g or t; T lymphocyte cDNA
gtgggtccaagtctttgtttgnnctaagatttgtnngctctcagacngtgtaaaacaaaatttattcatgttttctgcatattaaaaaatcttattgtaccaactggtaaactatt

SEQ ID NO: 33 - DNA, 210 bp, Homo sapiens
  unsure (25), (33), (42), (43), (58), (61): n = a or c or g or t; T lymphocyte cDNA
tgtctccaggatctcatgagccgcnacgtgttnagagggtcnncatcatacggggganggntggggcaaatcgccacctgtacctttcctctggccctgctgcccccacacccaactccganggcccacgctggggaaagcgggaagcgctcgctccctttcccccattagtgctctctctgcctggatcccggcagaagctatgaaagg

SEQ ID NO: 34 - DNA, 155 bp, Homo sapiens
  unsure (3), (5), (8), (17), (30), (36), (39), (49), (51),(71), (99), (104), (111): n = a or c or g or t; T lymphocyte cDNA
tancntgntacactcgntaaagaagagcangatcangcnactatactanangttagcatcactaacgcccncgcatgtgcatgaaacaccttctctgcncgccnattccanatttacactgggagaggtgccagcaactgaataaatacctctta

SEQ ID NO: 35 - DNA, 19 bp, Artificial Sequence
  5′ sequence for primer
ctctcaaggatctaccgct

SEQ ID NO: 36 - DNA, 20 bp, Artificial Sequence
  5′ sequence for primer
cagggtagacgacgctacgc

SEQ ID NO: 37 - DNA, 20 bp, Artificial Sequence
  5′ sequence for primer
taataccgcgccacatagca

SEQ ID NO: 38 - DNA, 55 bp, Artificial Sequence
  variation (55): v = a or c or g
  1-base anchored oligo(dT) primer
acgtaatacgactcactatagggcgaattgggtcgactttttttttttttttttv

SEQ ID NO: 39 - DNA, 40 bp, Artificial Sequence
  2-base anchored oligo(dT) primer, RP5.0
ctctcaaggatcttaccgctttttttttttttttttttat

SEQ ID NO: 40 - DNA, 40 bp, Artificial Sequence
  2-base anchored oligo(dT) primer, RP6.0
taataccgcgccacatagcattttttttttttttttttcg

SEQ ID NO: 41 - DNA, 40 bp, Artificial Sequence
  2-base anchored oligo(dT) primer, RP9.2
cagggtagacgacgctacgcttttttttttttttttttga

SEQ ID NO: 42 - DNA, 25 bp, Artificial Sequence
  adapter oligonucleotide A1
tagcgtccggcgcagcgacggccag

SEQ ID NO: 43 - DNA, 29 bp, Artificial Sequence
  adapter oligonucleotide A2
gatcctggccgtcggctgtctgtcggcgc

SEQ ID NO: 44 - DNA, 40 bp, Artificial Sequence
  variation (39), (40): v = a or c or g; n = a or c or g or t
  PCR primer
tgaagccgagacgtcggtcgttttttttttttttttttvn